SEQUENCE LISTING (50 sequences)

SEQ ID NO: 1 - DNA, 455 bp, Homo sapiens
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 2 - DNA, 899 bp, Adenovirus
acaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtg

SEQ ID NO: 3 - DNA, 1823 bp, Adenovirus
ctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggc

SEQ ID NO: 4 - DNA, 605 bp, Artificial
  synthetic DNA
tgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgcca

SEQ ID NO: 5 - RNA, 23 nt, Artificial
  synthetic RNA
uguaguguuuccuacuuuaugga

SEQ ID NO: 6 - RNA, 21 nt, Artificial
  synthetic RNA
cauaaaguagaaagcacuacu

SEQ ID NO: 7 - RNA, 22 nt, Artificial
  synthetic RNA
uagcagcacauaaugguuugug

SEQ ID NO: 8 - RNA, 22 nt, Artificial
  synthetic RNA
caggccauauugugcugccuca

SEQ ID NO: 9 - RNA, 22 nt, Artificial
  synthetic RNA
uagcagcacguaaauauuggcg

SEQ ID NO: 10 - RNA, 22 nt, Artificial
  synthetic RNA
ccaguauuaacugugcugcuga

SEQ ID NO: 11 - RNA, 22 nt, Artificial
  synthetic RNA
uagcuuaucagacugauguuga

SEQ ID NO: 12 - RNA, 21 nt, Artificial
  synthetic RNA
caacaccagucgaugggcugu

SEQ ID NO: 13 - RNA, 22 nt, Artificial
  synthetic RNA
ucguaccgugaguaauaaugcg

SEQ ID NO: 14 - RNA, 21 nt, Artificial
  synthetic RNA
cauuauuacuuuugguacgcg

SEQ ID NO: 15 - RNA, 23 nt, Artificial
  synthetic RNA
aacauucaacgcugucggugagu

SEQ ID NO: 16 - RNA, 22 nt, Artificial
  synthetic RNA
ugucaguuugucaaauacccca

SEQ ID NO: 17 - RNA, 22 nt, Artificial
  synthetic RNA
cguguauuugacaagcugaguu

SEQ ID NO: 18 - RNA, 22 nt, Artificial
  synthetic RNA
gaggguuggguggaggcucucc

SEQ ID NO: 19 - RNA, 21 nt, Artificial
  synthetic RNA
agggcccccccucaauccugu

SEQ ID NO: 20 - RNA, 24 nt, Artificial
  synthetic RNA
ucccugagacccuuuaaccuguga

SEQ ID NO: 21 - RNA, 21 nt, Artificial
  synthetic RNA
ugagaugaagcacuguagcuc

SEQ ID NO: 22 - RNA, 22 nt, Artificial
  synthetic RNA
ggugcagugcugcaucucuggu

SEQ ID NO: 23 - RNA, 23 nt, Artificial
  synthetic RNA
guccaguuuucccaggaaucccu

SEQ ID NO: 24 - RNA, 22 nt, Artificial
  synthetic RNA
ggauuccuggaaauacuguucu

SEQ ID NO: 25 - RNA, 23 nt, Artificial
  synthetic RNA
cccaguguucagacuaccuguuc

SEQ ID NO: 26 - RNA, 22 nt, Artificial
  synthetic RNA
ugagguaguagguuguauaguu

SEQ ID NO: 27 - DNA, 69 bp, Artificial
  synthetic DNA
gcggcctccataaagtaggaaacactacacagctccataaagtaggaaacactacattataagcggtac

SEQ ID NO: 28 - DNA, 113 bp, Artificial
  synthetic DNA
ggcctccataaagtaggaaacactacacagctccataaagtaggaaacactacattaattccataaagtaggaaacactacaccactccataaagtaggaaacactacagtac

SEQ ID NO: 29 - DNA, 1307 bp, Homo sapiens
accgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcgactcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 30 - DNA, 837 bp, Homo sapiens
gaggactccgcgacggtccgcaccctgcggccagagcggctttgagctcggctgcttccgcgctaggcgctttttcccagaagcaatccaggcgcgcccgctggttcttgagcgccaggaaaagcccggagctaacgaccggccgctcggcactgcacggggccccaagccgcagaagaaggacgacgggagggtaatgaagctgagcccaggtctcctaggaaggagagagtgcgccggagcagcgtgggaaagaagggaagagtgtcgttaagtttacggccaacggtggattatccgggccgctgcgcgtctgggggctgcggaatgcgcgaggagaacaagggcatgcccagtgggggcggcagcgatgagggtctggccacgccggcgcggggactagtggagaaggtgcgacactcctgggaagccggcgcggatcccaacggagtcaaccgtttcgggaggcgcgcgatccaggtcatgatgatgggcagcgcccgcgtggcggagctgctgctgctccacggcgcggagcccaactgcgcagaccctgccactctcacccgaccggtgcatgatgctgcccgggagggcttcctggacacgctggtggtgctgcaccgggccggggcgcggctggacgtgcgcgatgcctggggtcgtctgcccgtggacttggccgaggagcggggccaccgcgacgttgcagggtacctgcgcacagccacgggggactgacgccaggttccccagccgcccacaacgactttattttcttacccaatttcccacccccacccacctaattcgatgaaggctgccaacggggagcgg

SEQ ID NO: 31 - DNA, 987 bp, Homo sapiens
cggagagggggagaacagacaacgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatataagccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactc

SEQ ID NO: 32 - DNA, 8972 bp, Homo sapiens
gtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtacgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcaagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggagcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcgtcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggacttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccagtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcacccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgt

SEQ ID NO: 33 - DNA, 5711 bp, Homo sapiens
agctcgctgagacttcctggaccccgcaccaggctgtggggtttctcagataactgggcccctgcgctcaggaggccttcaccctctgctctgggtaaagttcattggaacagaaagaaatggatttatctgctcttcgcgttgaagaagtacaaaatgtcattaatgctatgcagaaaatcttagagtgtcccatctgtctggagttgatcaaggaacctgtctccacaaagtgtgaccacatattttgcaaattttgcatgctgaaacttctcaaccagaagaaagggccttcacagtgtcctttatgtaagaatgatataaccaaaaggagcctacaagaaagtacgagatttagtcaacttgttgaagagctattgaaaatcatttgtgcttttcagcttgacacaggtttggagtatgcaaacagctataattttgcaaaaaaggaaaataactctcctgaacatctaaaagatgaagtttctatcatccaaagtatgggctacagaaaccgtgccaaaagacttctacagagtgaacccgaaaatccttccttgcaggaaaccagtctcagtgtccaactctctaaccttggaactgtgagaactctgaggacaaagcagcggatacaacctcaaaagacgtctgtctacattgaattgggatctgattcttctgaagataccgttaataaggcaacttattgcagtgtgggagatcaagaattgttacaaatcacccctcaaggaaccagggatgaaatcagtttggattctgcaaaaaaggctgcttgtgaattttctgagacggatgtaacaaatactgaacatcatcaacccagtaataatgatttgaacaccactgagaagcgtgcagctgagaggcatccagaaaagtatcagggtagttctgtttcaaacttgcatgtggagccatgtggcacaaatactcatgccagctcattacagcatgagaacagcagtttattactcactaaagacagaatgaatgtagaaaaggctgaattctgtaataaaagcaaacagcctggcttagcaaggagccaacataacagatgggctggaagtaaggaaacatgtaatgataggcggactcccagcacagaaaaaaaggtagatctgaatgctgatcccctgtgtgagagaaaagaatggaataagcagaaactgccatgctcagagaatcctagagatactgaagatgttccttggataacactaaatagcagcattcagaaagttaatgagtggttttccagaagtgatgaactgttaggttctgatgactcacatgatggggagtctgaatcaaatgccaaagtagctgatgtattggacgttctaaatgaggtagatgaatattctggttcttcagagaaaatagacttactggccagtgatcctcatgaggctttaatatgtaaaagtgaaagagttcactccaaatcagtagagagtaatattgaagacaaaatatttgggaaaacctatcggaagaaggcaagcctccccaacttaagccatgtaactgaaaatctaattataggagcatttgttactgagccacagataatacaagagcgtcccctcacaaataaattaaagcgtaaaaggagacctacatcaggccttcatcctgaggattttatcaagaaagcagatttggcagttcaaaagactcctgaaatgataaatcagggaactaaccaaacggagcagaatggtcaagtgatgaatattactaatagtggtcatgagaataaaacaaaaggtgattctattcagaatgagaaaaatcctaacccaatagaatcactcgaaaaagaatctgctttcaaaacgaaagctgaacctataagcagcagtataagcaatatggaactcgaattaaatatccacaattcaaaagcacctaaaaagaataggctgaggaggaagtcttctaccaggcatattcatgcgcttgaactagtagtcagtagaaatctaagcccacctaattgtactgaattgcaaattgatagttgttctagcagtgaagagataaagaaaaaaaagtacaaccaaatgccagtcaggcacagcagaaacctacaactcatggaaggtaaagaacctgcaactggagccaagaagagtaacaagccaaatgaacagacaagtaaaagacatgacagcgatactttcccagagctgaagttaacaaatgcacctggttcttttactaagtgttcaaataccagtgaacttaaagaatttgtcaatcctagccttccaagagaagaaaaagaagagaaactagaaacagttaaagtgtctaataatgctgaagaccccaaagatctcatgttaagtggagaaagggttttgcaaactgaaagatctgtagagagtagcagtatttcattggtacctggtactgattatggcactcaggaaagtatctcgttactggaagttagcactctagggaaggcaaaaacagaaccaaataaatgtgtgagtcagtgtgcagcatttgaaaaccccaagggactaattcatggttgttccaaagataatagaaatgacacagaaggctttaagtatccattgggacatgaagttaaccacagtcgggaaacaagcatagaaatggaagaaagtgaacttgatgctcagtatttgcagaatacattcaaggtttcaaagcgccagtcatttgctccgttttcaaatccaggaaatgcagaagaggaatgtgcaacattctctgcccactctgggtccttaaagaaacaaagtccaaaagtcacttttgaatgtgaacaaaaggaagaaaatcaaggaaagaatgagtctaatatcaagcctgtacagacagttaatatcactgcaggctttcctgtggttggtcagaaagataagccagttgataatgccaaatgtagtatcaaaggaggctctaggttttgtctatcatctcagttcagaggcaacgaaactggactcattactccaaataaacatggacttttacaaaacccatatcgtataccaccactttttcccatcaagtcatttgttaaaactaaatgtaagaaaaatctgctagaggaaaactttgaggaacattcaatgtcacctgaaagagaaatgggaaatgagaacattccaagtacagtgagcacaattagccgtaataacattagagaaaatgtttttaaagaagccagctcaagcaatattaatgaagtaggttccagtactaatgaagtgggctccagtattaatgaaataggttccagtgatgaaaacattcaagcagaactaggtagaaacagagggccaaaattgaatgctatgcttagattaggggttttgcaacctgaggtctataaacaaagtcttcctggaagtaattgtaagcatcctgaaataaaaaagcaagaatatgaagaagtagttcagactgttaatacagatttctctccatatctgatttcagataacttagaacagcctatgggaagtagtcatgcatctcaggtttgttctgagacacctgatgacctgttagatgatggtgaaataaaggaagatactagttttgctgaaaatgacattaaggaaagttctgctgtttttagcaaaagcgtccagaaaggagagcttagcaggagtcctagccctttcacccatacacatttggctcagggttaccgaagaggggccaagaaattagagtcctcagaagagaacttatctagtgaggatgaagagcttccctgcttccaacacttgttatttggtaaagtaaacaatataccttctcagtctactaggcatagcaccgttgctaccgagtgtctgtctaagaacacagaggagaatttattatcattgaagaatagcttaaatgactgcagtaaccaggtaatattggcaaaggcatctcaggaacatcaccttagtgaggaaacaaaatgttctgctagcttgttttcttcacagtgcagtgaattggaagacttgactgcaaatacaaacacccaggatcctttcttgattggttcttccaaacaaatgaggcatcagtctgaaagccagggagttggtctgagtgacaaggaattggtttcagatgatgaagaaagaggaacgggcttggaagaaaataatcaagaagagcaaagcatggattcaaacttaggtgaagcagcatctgggtgtgagagtgaaacaagcgtctctgaagactgctcagggctatcctctcagagtgacattttaaccactcagcagagggataccatgcaacataacctgataaagctccagcaggaaatggctgaactagaagctgtgttagaacagcatgggagccagccttctaacagctacccttccatcataagtgactcttctgcccttgaggacctgcgaaatccagaacaaagcacatcagaaaaagcagtattaacttcacagaaaagtagtgaataccctataagccagaatccagaaggcctttctgctgacaagtttgaggtgtctgcagatagttctaccagtaaaaataaagaaccaggagtggaaaggtcatccccttctaaatgcccatcattagatgataggtggtacatgcacagttgctctgggagtcttcagaatagaaactacccatctcaagaggagctcattaaggttgttgatgtggaggagcaacagctggaagagtctgggccacacgatttgacggaaacatcttacttgccaaggcaagatctagagggaaccccttacctggaatctggaatcagcctcttctctgatgaccctgaatctgatccttctgaagacagagccccagagtcagctcgtgttggcaacataccatcttcaacctctgcattgaaagttccccaattgaaagttgcagaatctgcccagagtccagctgctgctcatactactgatactgctgggtataatgcaatggaagaaagtgtgagcagggagaagccagaattgacagcttcaacagaaagggtcaacaaaagaatgtccatggtggtgtctggcctgaccccagaagaatttatgctcgtgtacaagtttgccagaaaacaccacatcactttaactaatctaattactgaagagactactcatgttgttatgaaaacagatgctgagtttgtgtgtgaacggacactgaaatattttctaggaattgcgggaggaaaatgggtagttagctatttctgggtgacccagtctattaaagaaagaaaaatgctgaatgagcatgattttgaagtcagaggagatgtggtcaatggaagaaaccaccaaggtccaaagcgagcaagagaatcccaggacagaaagatcttcagggggctagaaatctgttgctatgggcccttcaccaacatgcccacagatcaactggaatggatggtacagctgtgtggtgcttctgtggtgaaggagctttcatcattcacccttggcacaggtgtccacccaattgtggttgtgcagccagatgcctggacagaggacaatggcttccatgcaattgggcagatgtgtgaggcacctgtggtgacccgagagtgggtgttggacagtgtagcactctaccagtgccaggagctggacacctacctgataccccagatcccccacagccactactga

SEQ ID NO: 34 - DNA, 2680 bp, Homo sapiens
ggttatcctgaatacatgtctaacaattttccttgcaacgttagctgttgtttttcactgtttccaaaggatcaaaattgcttcagaaattggagacatatttgatttaaaaggaaaaacttgaacaaatggacaatatgtctattacgaatacaccaacaagtaatgatgcctgtctgagcattgtgcatagtttgatgtgccatagacaaggtggagagagtgaaacatttgcaaaaagagcaattgaaagtttggtaaagaagctgaaggagaaaaaagatgaattggattctttaataacagctataactacaaatggagctcatcctagtaaatgtgttaccatacagagaacattggatgggaggcttcaggtggctggtcggaaaggatttcctcatgtgatctatgcccgtctctggaggtggcctgatcttcacaaaaatgaactaaaacatgttaaatattgtcagtatgcgtttgacttaaaatgtgatagtgtctgtgtgaatccatatcactacgaacgagttgtatcacctggaattgatctctcaggattaacactgcagagtaatgctccatcaagtatgatggtgaaggatgaatatgtgcatgactttgagggacagccatcgttgtccactgaaggacattcaattcaaaccatccagcatccaccaagtaatcgtgcatcgacagagacatacagcaccccagctctgttagccccatctgagtctaatgctaccagcactgccaactttcccaacattcctgtggcttccacaagtcagcctgccagtatactggggggcagccatagtgaaggactgttgcagatagcatcagggcctcagccaggacagcagcagaatggatttactggtcagccagctacttaccatcataacagcactaccacctggactggaagtaggactgcaccatacacacctaatttgcctcaccaccaaaacggccatcttcagcaccacccgcctatgccgccccatcccggacattactggcctgttcacaatgagcttgcattccagcctcccatttccaatcatcctgctcctgagtattggtgttccattgcttactttgaaatggatgttcaggtaggagagacatttaaggttccttcaagctgccctattgttactgttgatggatacgtggacccttctggaggagatcgcttttgtttgggtcaactctccaatgtccacaggacagaagccattgagagagcaaggttgcacataggcaaaggtgtgcagttggaatgtaaaggtgaaggtgatgtttgggtcaggtgccttagtgaccacgcggtctttgtacagagttactacttagacagagaagctgggcgtgcacctggagatgctgttcataagatctacccaagtgcatatataaaggtctttgatttgcgtcagtgtcatcgacagatgcagcagcaggcggctactgcacaagctgcagcagctgcccaggcagcagccgtggcaggaaacatccctggcccaggatcagtaggtggaatagctccagctatcagtctgtcagctgctgctggaattggtgttgatgaccttcgtcgcttatgcatactcaggatgagttttgtgaaaggctggggaccggattacccaagacagagcatcaaagaaacaccttgctggattgaaattcacttacaccgggccctccagctcctagacgaagtacttcataccatgccgattgcagacccacaacctttagactgaggtcttttaccgttggggcccttaaccttatcaggatggtggactacaaaatacaatcctgtttataatctgaagatatatttcacttttcttctgctttatcttttcataaagggttgaaaatgtgtttgctgccttgctcctagcagacagaaactggattaaaacaatttttttttcctcttcagaacttgtcaggcatggctcagagcttgaagattaggagaaacacattcttattaattcttcacctgttatgtatgaaggaatcattccagtgctagaaaatttagccctttaaaacgtcttagagccttttatctgcagaacatcgatatgtatatcattctacagaataatccagtattgctgattttaaaggcagagaagttctcaaagttaattcacctatgttattttgtgtacaagttgttattgttgaacatacttcaaaaataatgtgccatgtgggtgagttaattttaccaagagtaactttactctgtgtttaaaaatgaagttaataatgtattgtaatctttcatccaaaatattttttgcaagttatattagtgaagatggtttcaattcagattgtcttgcaacttcagttttatttttgccaaggcaaaaaactcttaatctgtgtgtatattgagaatcccttaaaattaccagacaaaaaaatttaaaattacgtttgttattcctagtggatgactgttgatgaagtatacttttcccctgttaaacagtagttgtattcttctgtatttctaggcacaaggttggttgctaagaagcctataagaggaatttcttttccttcattcatagggaaaggttttgtattttttaaaacactaaaagcagcgtcactctacctaatgtct

SEQ ID NO: 35 - DNA, 1095 bp, Homo sapiens
tccccgctctgctctgtccggtcacaggactttttgccctctgttcccgggtccctcaggcggccacccagtgggcacactcccaggcggcgctccggccccgcgctccctccctctgcctttcattcccagctgtcaacatcctggaagctttgaagctcaggaaagaagagaaatccactgagaacagtctgtaaaggtccgtagtgctatctacatccagacggtggaagggagagaaagagaaagaaggtatcctaggaatacctgcctgcttagaccctctataaaagctctgtgcatcctgccactgaggactccgaagaggtagcagtcttctgaaagacttcaactgtgaggacatgtcgttcagatttggccaacatctcatcaagccctctgtagtgtttctcaaaacagaactgtccttcgctcttgtgaataggaaacctgtggtaccaggacatgtccttgtgtgcccgctgcggccagtggagcgcttccatgacctgcgtcctgatgaagtggccgatttgtttcagacgacccagagagtcgggacagtggtggaaaaacatttccatgggacctctctcaccttttccatgcaggatggccccgaagccggacagactgtgaagcacgttcacgtccatgttcttcccaggaaggctggagactttcacaggaatgacagcatctatgaggagctccagaaacatgacaaggaggactttcctgcctcttggagatcagaggaggaaatggcagcagaagccgcagctctgcgggtctactttcagtgacacagatgtttttcagatcctgaattccagcaaaagagctattgccaaccagtttgaagaccgcccccccgcctctccccaagaggaactgaatcagcatgaaaatgcagtttcttcatctcaccatcctgtattcttcaaccagtgatcccccacctcggtcactccaactcccttaaaatacctagacctaaacggctcagacaggcagatttgaggtttccccctgtctccttattcggcagccttatgattaaacttccttctctgctgcaaaaaaaaaaaaaaa

SEQ ID NO: 36 - DNA, 2234 bp, Homo sapiens
aggggacgcagcgaaaccggggcccgcgccaggccagccgggacggacgccgatgcccggggctgcgacggctgcagagcgagctgccctcggaggccggcgtggggaagatggcccagtccaccgccacctcccctgatgggggcaccacgtttgagcacctctggagctctctggaaccagacagcacctacttcgaccttccccagtcaagccgggggaataatgaggtggtgggcggaacggattccagcatggacgtcttccacctggagggcatgactacatctgtcatggcccagttcaatctgctgagcagcaccatggaccagatgagcagccgcgcggcctcggccagcccctacaccccagagcacgccgccagcgtgcccacccactcgccctacgcacaacccagctccaccttcgacaccatgtcgccggcgcctgtcatcccctccaacaccgactaccccggaccccaccactttgaggtcactttccagcagtccagcacggccaagtcagccacctggacgtactccccgctcttgaagaaactctactgccagatcgccaagacatgccccatccagatcaaggtgtccaccccgccacccccaggcactgccatccgggccatgcctgtttacaagaaagcggagcacgtgaccgacgtcgtgaaacgctgccccaaccacgagctcgggagggacttcaacgaaggacagtctgctccagccagccacctcatccgcgtggaaggcaataatctctcgcagtatgtggatgaccctgtcaccggcaggcagagcgtcgtggtgccctatgagccaccacaggtggggacggaattcaccaccatcctgtacaacttcatgtgtaacagcagctgtgtagggggcatgaaccggcggcccatcctcatcatcatcaccctggagatgcgggatgggcaggtgctgggccgccggtcctttgagggccgcatctgcgcctgtcctggccgcgaccgaaaagctgatgaggaccactaccgggagcagcaggccctgaacgagagctccgccaagaacggggccgccagcaagcgtgccttcaagcagagcccccctgccgtccccgcccttggtgccggtgtgaagaagcggcggcatggagacgaggacacgtactaccttcaggtgcgaggccgggagaactttgagatcctgatgaagctgaaagagagcctggagctgatggagttggtgccgcagccactggtggactcctatcggcagcagcagcagctcctacagaggccgagtcacctacagcccccgtcctacgggccggtcctctcgcccatgaacaaggtgcacgggggcatgaacaagctgccctccgtcaaccagctggtgggccagcctcccccgcacagttcggcagctacacccaacctggggcccgtgggccccgggatgctcaacaaccatggccacgcagtgccagccaacggcgagatgagcagcagccacagcgcccagtccatggtctcggggtcccactgcactccgccacccccctaccacgccgaccccagcctcgtcagttttttaacaggattggggtgtccaaactgcatcgagtatttcacctcccaagggttacagagcatttaccacctgcagaacctgaccattgaggacctgggggccctgaagatccccgagcagtaccgcatgaccatctggcggggcctgcaggacctgaagcagggccacgactacagcaccgcgcagcagctgctccgctctagcaacgcggccaccatctccatcggcggctcaggggaactgcagcgccagcgggtcatggaggccgtgcacttccgcgtgcgccacaccatcaccatccccaaccgcggcggcccaggcggcggccctgacgagtgggcggacttcggcttcgacctgcccgactgcaaggcccgcaagcagcccatcaaggaggagttcacggaggccgagatccactgagggcctcgcctggctgcagcctgcgccaccgcccagagacccaagctgcctcccctctccttcctgtgtgtccaaaactgcctcaggaggcaggaccttcgggctgtgcccggggaaaggcaaggtccggcccatccccaggcacctcacaggccccaggaaaggcccagccaccgaagccgcctgtggacagcctgagtcacctgcagaacc

SEQ ID NO: 37 - DNA, 4344 bp, Homo sapiens
atggcctcggctggtaacgccgccgagccccaggaccgcggcggcggcggcagcggctgtatcggtgccccgggacggccggctggaggcgggaggcgcagacggacgggggggctgcgccgtgctgccgcgccggaccgggactatctgcaccggcccagctactgcgacgccgccttcgctctggagcagatttccaaggggaaggctactggccggaaagcgccactgtggctgagagcgaagtttcagagactcttatttaaactgggttgttacattcaaaaaaactgcggcaagttcttggttgtgggcctcctcatatttggggccttcgcggtgggattaaaagcagcgaacctcgagaccaacgtggaggagctgtgggtggaagttggaggacgagtaagtcgtgaattaaattatactcgccagaagattggagaagaggctatgtttaatcctcaactcatgatacagacccctaaagaagaaggtgctaatgtcctgaccacagaagcgctcctacaacacctggactcggcactccaggccagccgtgtccatgtatacatgtacaacaggcagtggaaattggaacatttgtgttacaaatcaggagagcttatcacagaaacaggttacatggatcagataatagaatatctttacccttgtttgattattacacctttggactgcttctgggaaggggcgaaattacagtctgggacagcatacctcctaggtaaacctcctttgcggtggacaaacttcgaccctttggaattcctggaagagttaaagaaaataaactatcaagtggacagctgggaggaaatgctgaataaggctgaggttggtcatggttacatggaccgcccctgcctcaatccggccgatccagactgccccgccacagcccccaacaaaaattcaaccaaacctcttgatatggcccttgttttgaatggtggatgtcatggcttatccagaaagtatatgcactggcaggaggagttgattgtgggtggcacagtcaagaacagcactggaaaactcgtcagcgcccatgccctgcagaccatgttccagttaatgactcccaagcaaatgtacgagcacttcaaggggtacgagtatgtctcacacatcaactggaacgaggacaaagcggcagccatcctggaggcctggcagaggacatatgtggaggtggttcatcagagtgtcgcacagaactccactcaaaaggtgctttccttcaccaccacgaccctggacgacatcctgaaatccttctctgacgtcagtgtcatccgcgtggccagcggctacttactcatgctcgcctatgcctgtctaaccatgctgcgctgggactgctccaagtcccagggtgccgtggggctggctggcgtcctgctggttgcactgtcagtggctgcaggactgggcctgtgctcattgatcggaatttcctttaacgctgcaacaactcaggttttgccatttctcgctcttggtgttggtgtggatgatgtttttcttctggcccacgccttcagtgaaacaggacagaataaaagaatcccttttgaggacaggaccggggagtgcctgaagcgcacaggagccagcgtggccctcacgtccatcagcaatgtcacagccttcttcatggccgcgttaatcccaattcccgctctgcgggcgttctccctccaggcagcggtagtagtggtgttcaattttgccatggttctgctcatttttcctgcaattctcagcatggatttatatcgacgcgaggacaggagactggatattttctgctgttttacaagcccctgcgtcagcagagtgattcaggttgaacctcaggcctacaccgacacacacgacaatacccgctacagccccccacctccctacagcagccacagctttgcccatgaaacgcagattaccatgcagtccactgtccagctccgcacggagtacgacccccacacgcacgtgtactacaccaccgctgagccgcgctccgagatctctgtgcagcccgtcaccgtgacacaggacaccctcagctgccagagcccagagagcaccagctccacaagggacctgctctcccagttctccgactccagcctccactgcctcgagcccccctgtacgaagtggacactctcatcttttgctgagaagcactatgctcctttcctcttgaaaccaaaagccaaggtagtggtgatcttcctttttctgggcttgctgggggtcagcctttatggcaccacccgagtgagagacgggctggaccttacggacattgtacctcgggaaaccagagaatatgactttattgctgcacaattcaaatacttttctttctacaacatgtatatagtcacccagaaagcagactacccgaatatccagcacttactttacgacctacacaggagtttcagtaacgtgaagtatgtcatgttggaagaaaacaaacagcttcccaaaatgtggctgcactacttcagagactggcttcagggacttcaggatgcatttgacagtgactgggaaaccgggaaaatcatgccaaacaattacaagaatggatcagacgatggagtccttgcctacaaactcctggtgcaaaccggcagccgcgataagcccatcgacatcagccagttgactaaacagcgtctggtggatgcagatggcatcattaatcccagcgctttctacatctacctgacggcttgggtcagcaacgaccccgtcgcgtatgctgcctcccaggccaacatccggccacaccgaccagaatgggtccacgacaaagccgactacatgcctgaaacaaggctgagaatcccggcagcagagcccatcgagtatgcccagttccctttctacctcaacgggttgcgggacacctcagactttgtggaggcaattgaaaaagtaaggaccatctgcagcaactatacgagcctggggctgtccagttaccccaacggctaccccttcctcttctgggagcagtacatcggcctccgccactggctgctgctgttcatcagcgtggtgttggcctgcacattcctcgtgtgcgctgtcttccttctgaacccctggacggccgggatcattgtgatggtcctggcgctgatgacggtcgagctgttcggcatgatgggcctcatcggaatcaagctcagtgccgtgcccgtggtcatcctgatcgcttctgttggcataggagtggagttcaccgttcacgttgctttggcctttctgacggccatcggcgacaagaaccgcagggctgtgcttgccctggagcacatgtttgcacccgtcctggatggcgccgtgtccactctgctgggagtgctgatgctggcgggatctgagttcgacttcattgtcaggtatttctttgctgtgctggcgatcctcaccatcctcggcgttctcaatgggctggttttgcttcccgtgcttttgtctttctttggaccatatcctgaggtgtctccagccaacggcttgaaccgcctgcccacaccctcccctgagccaccccccagcgtggtccgcttcgccatgccgcccggccacacgcacagcgggtctgattcctccgactcggagtatagttcccagacgacagtgtcaggcctcagcgaggagcttcggcactacgaggcccagcagggcgcgggaggccctgcccaccaagtgatcgtggaagccacagaaaaccccgtcttcgcccactccactgtggtccatcccgaatccaggcatcacccaccctcgaacccgagacagcagccccacctggactcagggtccctgcctcccggacggcaaggccagcagccccgcagggacccccccagagaaggcttgtggccacccctctacagaccgcgcagagacgcttttgaaatttctactgaagggcattctggccctagcaatagggcccgctggggccctcgcggggcccgttctcacaaccctcggaacccagcgtccactgccatgggcagctccgtgcccggctactgccagcccatcaccactgtgacggcttctgcctccgtgactgtcgccgtgcacccgccgcctgtccctgggcctgggcggaacccccgagggggactctgcccaggctaccctgagactgaccacggcctgtttgaggacccccacgtgcctttccacgtccggtgtgagaggagggattcgaaggtggaagtcattgagctgcaggacgtggaatgcgaggagaggccccggggaagcagctccaactga

SEQ ID NO: 38 - DNA, 4740 bp, Homo sapiens
ttccggtttttctcaggggacgttgaaattatttttgtaacgggagtcgggagaggacggggcgtgccccgcgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcacgagttgacctagatgagatgtcgttcactttactgagctacagaaaaacatacgaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatcgaagattatttttggatcatgataaaactcttcagactgattctatagacagttttgaaacacagagaacaccacgaaaaagtaaccttgatgaagaggtgaatataattcctccacacactccagttaggactgttatgaacactatccaacaattaatgatgattttaaattctgcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgagtcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtc

SEQ ID NO: 39 - DNA, 4608 bp, Homo sapiensl
atggagaatagtcttagatgtgtttgggtacccaagctggcttttgtactcttcggagcttccttgctcagcgcgcatcttcaagtaaccggttttcaaattaaagctttcacagcactgcgcttcctctcagaaccttctgatgccgtcacaatgcggggaggaaatgtcctcctcgactgctccgcggagtccgaccgaggagttccagtgatcaagtggaagaaagatggcattcatctggccttgggaatggatgaaaggaagcagcaactttcaaatgggtctctgctgatacaaaacatacttcattccagacaccacaagccagatgagggactttaccaatgtgaggcatctttaggagattctggctcaattattagtcggacagcaaaagttgcagtagcaggaccactgaggttcctttcacagacagaatctgtcacagccttcatgggagacacagtgctactcaagtgtgaagtcattggggagcccatgccaacaatccactggcagaagaaccaacaagacctgactccaatcccaggtgactcccgagtggtggtcttgccctctggagcattgcagatcagccgactccaaccgggggacattggaatttaccgatgctcagctcgaaatccagccagctcaagaacaggaaatgaagcagaagtcagaattttatcagatccaggactgcatagacagctgtattttctgcaaagaccatccaatgtagtagccattgaaggaaaagatgctgtcctggaatgttgtgtttctggctatcctccaccaagttttacctggttacgaggcgaggaagtcatccaactcaggtctaaaaagtattctttattgggtggaagcaacttgcttatctccaatgtgacagatgatgacagtggaatgtatacctgtgttgtcacatataaaaatgagaatattagtgcctctgcagagctcacagtcttggttccgccatggtttttaaatcatccttccaacctgtatgcctatgaaagcatggatattgagtttgaatgtacagtctctggaaagcctgtgcccactgtgaattggatgaagaatggagatgtggtcattcctagtgattattttcagatagtgggaggaagcaacttacggatacttggggtggtgaagtcagatgaaggcttttatcaatgtgtggctgaaaatgaggctggaaatgcccagaccagtgcacagctcattgtccctaagcctgcaatcccaagctccagtgtcctcccttcggctcccagagatgtggtccctgtcttggtttccagccgatttgtccgtctcagctggcgcccacctgcagaagcgaaagggaacattcaaactttcacggtctttttctccagagaaggtgacaacagggaacgagcattgaatacaacacagcctgggtcccttcagctcactgtgggaaacctgaagccagaagccatgtacacctttcgagttgtggcttacaatgaatggggaccgggagagagttctcaacccatcaaggtggccacacagcctgagttgcaagttccagggccagtagaaaacctgcaagctgtatctacctcacctacctcaattcttattacctgggaaccccctgcctatgcaaacggtccagtccaaggttacagattgttctgcactgaggtgtccacaggaaaagaacagaatatagaggttgatggactatcttataaactggaaggcctgaaaaaattcaccgaatatagtcttcgattcttagcttataatcgctatggtccgggcgtctctactgatgatataacagtggttacactttctgacgtgccaagtgccccgcctcagaacgtctccctggaagtggtcaattcaagaagtatcaaagttagctggctgcctcctccatcaggaacacaaaatggatttattaccggctataaaattcgacacagaaagacgacccgcaggggtgagatggaaacactggagccaaacaacctctggtacctattcacaggactggagaaaggaagtcagtacagtttccaggtgtcagccatgacagtcaatggtactggaccaccttccaactggtatactgcagagactccagagaatgatctagatgaatctcaagttcctgatcaaccaagctctcttcatgtgaggccccagactaactgcatcatcatgagttggactcctcccttgaacccaaacatcgtggtgcgaggttatattatcggttatggcgttgggagcccttacgctgagacagtgcgtgtggacagcaagcagcgatattattccattgagaggttagagtcaagttcccattatgtaatctccctaaaagcttttaacaatgccggagaaggagttcctctttatgaaagtgccaccaccaggtctataaccgatcccactgacccagttgattattatcctttgcttgatgatttccccacctcggtcccagatctctccacccccatgctcccaccagtaggtgtacaggctgtggctcttacccatgatgctgtgagggtcagctgggcagacaactctgtccctaagaaccaaaagacgtctgaggtgcgactttacaccgtccggtggagaaccagcttttctgcaagtgcaaaatacaagtcagaagacacaacatctctaagttacacagcaacaggcctcaaaccaaacacaatgtatgaattctcggtcatggtaacaaaaaacagaaggtccagtacttggagcatgactgcacatgccaccacgtatgaagcagcccccacctctgctcccaaggactttacagtcattactagggaagggaagcctcgtgccgtcattgtgagttggcagcctcccttggaagccaatgggaaaattactgcttacatcttattttataccttggacaagaacatcccaattgatgactggattatggaaacaatcagtggtgataggcttactcatcaaatcatggatctcaaccttgatactatgtattactttcgaattcaagcacgaaattcaaaaggagtggggccactctctgatcccatcctcttcaggactctgaaagtggaacaccctgacaaaatggctaatgaccaaggtcgtcatggagatggaggttattggccagttgatactaatttgattgatagaagcaccctaaatgagccgccaattggacaaatgcaccccccgcatggcagtgtcactcctcagaagaacagcaacctgcttgtgatcattgtggtcaccgttggtgtcatcacagtgctggtagtggtcatcgtggctgtgatttgcacccgacgctcttcagcccagcagagaaagaaacgggccacccacagtgctggcaaaaggaagggcagccagaaggacctccgaccccctgatctttggatccatcatgaagaaatggagatgaaaaatattgaaaagccatctggcactgaccctgcaggaagggactctcccatccaaagttgccaagacctcacaccagtcagccacagccagtcagaaacccaactgggaagcaaaagcacctctcattcaggtcaagacactgaggaagcagggagctctatgtccactctggagaggtcgctggctgcacgccgagccccccgggccaagctcatgattcccatggatgcccagtccaacaatcctgctgtcgtgagcgccatcccggtgccaacgctagaaagtgcccagtacccaggaatcctcccgtctcccacctgtggatatccccacccgcagttcactctccggcctgtgccattcccaacactctcagtggaccgaggtttcggagcaggaagaagtcagtcagtgagtgaaggaccaactacccaacaaccacctatgctgcccccatctcagcctgagcattctagcagcgaggaggcaccaagcagaaccatccccacagcttgtgttcgaccaactcacccactccgcagctttgctaatcctttgctacctccaccaatgagtgcaatagaaccgaaagtcccttacacaccacttttgtctcagccagggcccactcttcctaagacccatgtgaaaacagcctcccttgggttggctggaaaagcaagatcccctttgcttcctgtgtctgtgccaacagcccctgaagtgtctgaggagagccacaaaccaacagaggattcagccaatgtgtatgaacaggatgatctgagtgaacaaatggcaagtttggaaggactcatgaagcagcttaatgccatcacaggctcagccttttaacatgtatttctgaatggatgaggtgaattttccgggaactttgcagcataccaattacccataaacagcacacctgtgtccaagaactctaaccagtgtacaggtcacccatcaggaccactcagttaaggaagatcctgaagcagttcagaaggaataagcattccttctttcacaggcatcaggaattgtcaaatgatgattatgagttccctaaacaaaagcaaagatgcattttcactgcaatgtcaaagtttagctgct

SEQ ID NO: 40 - DNA, 8959 bp, Homo sapiens
ccccagcctccttgccaacgccccctttccctctccccctcccgctcggcgctgaccccccatccccacccccgtgggaacactgggagcctgcactccacagaccctctccttgcctcttccctcacctcagcctccgctccccgccctcttcccggcccagggcgccggcccacccttccctccgccgccccccggccgcggggaggacatggccgcgcacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattagctcacaaatgcttttttacatctgcaagaaattaactagtcatcaaatgcttagtagcacagaaattctcaagtggttgcgggaaatattgatctgcaggaataaatttcttcttaaaaataagcaggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatggatagtgcagcaggatgcagcggaacccccccaatttgccgacaagcccagaccaaactagaagtggccctgtacatgtttctgtggaaccctgacactgaagctgttctggttgccatgtcctgtttccgccacctctgtgaggaagcagatatccggtgtggggtggatgaagtgtcagtgcataacctcttgcccaactataacacattcatggagtttgcctctgtcagcaatatgatgtcaacaggaagagcagcacttcagaaaagagtgatggcactgctgaggcgcattgagcatcccactgcaggaaacactgaggcttgggaagatacacatgcaaaatgggaacaagcaacaaagctaatccttaactatccaaaagccaaaatggaagatggccaggctgctgaaagccttcacaagaccattgttaagaggcgaatgtcccatgtgagtggaggaggatccatagatttgtctgacacagactccctacaggaatggatcaacatgactggcttcctttgtgcccttggaggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacaggttttattgactgataccaatactcaatttgtagaacaaaccatagctataatgaagaacttgctagataatcatactgaaggcagctctgaacatctagggcaagctagcattgaaacaatgatgttaaatctggtcaggtatgttcgtgtgcttgggaatatggtccatgcaattcaaataaaaacgaaactgtgtcaattagttgaagtaatgatggcaaggagagatgacctctcattttgccaagagatgaaatttaggaataagatggtagaatacctgacagactgggttatgggaacatcaaaccaagcagcagatgatgatgtaaaatgtcttacaagagatttggaccaggcaagcatggaagcagtagtttcacttctagctggtctccctctgcagcctgaagaaggagatggtgtggaattgatggaagccaaatcacagttatttcttaaatacttcacattatttatgaaccttttgaatgactgcagtgaagttgaagatgaaagtgcgcaaacaggtggcaggaaacgtggcatgtctcggaggctggcatcactgaggcactgtacggtccttgcaatgtcaaacttactcaatgccaacgtagacagtggtctcatgcactccataggcttaggttaccacaaggatctccagacaagagctacatttatggaagttctgacaaaaatccttcaacaaggcacagaatttgacacacttgcagaaacagtattggctgatcggtttgagagattggtggaactggtcacaatgatgggtgatcaaggagaactccctatagcgatggctctggccaatgtggttccttgttctcagtgggatgaactagctcgagttctggttactctgtttgattctcggcatttactctaccaactgctctggaacatgttttctaaagaagtagaattggcagactccatgcagactctcttccgaggcaacagcttggccagtaaaataatgacattctgtttcaaggtatatggtgctacctatctacaaaaactcctggatcctttattacgaattgtgatcacatcctctgattggcaacatgttagctttgaagtggatcctaccaggttagaaccatcagagagccttgaggaaaaccagcggaacctccttcagatgactgaaaagttcttccatgccatcatcagttcctcctcagaattcccccctcaacttcgaagtgtgtgccactgtttataccaggtggttagccagcgtttccctcagaacagcatcggtgcagtaggaagtgccatgttcctcagatttatcaatcctgccattgtctcaccgtatgaagcagggattttagataaaaagccaccacctagaatcgaaaggggcttgaagttaatgtcaaagatacttcagagtattgccaatcatgttctcttcacaaaagaagaacatatgcggcctttcaatgattttgtgaaaagcaactttgatgcagcacgcaggtttttccttgatatagcatctgattgtcctacaagtgatgcagtaaatcatagtctttccttcataagtgacggcaatgtgcttgctttacatcgtctactctggaacaatcaggagaaaattgggcagtatctttccagcaacagggatcataaagctgttggaagacgaccttttgataagatggcaacacttcttgcatacctgggtcctccagagcacaaacctgtggcagatacacactggtccagccttaaccttaccagttcaaagtttgaggaatttatgactaggcatcaggtacatgaaaaagaagaattcaaggctttgaaaacgttaagtattttctaccaagctgggacttccaaagctgggaatcctattttttattatgttgcacggaggttcaaaactggtcaaatcaatggtgatttgctgatataccatgtcttactgactttaaagccatattatgcaaagccatatgaaattgtagtggaccttacccataccgggcctagcaatcgctttaaaacagactttctctctaagtggtttgttgtttttcctggctttgcttacgacaacgtctccgcagtctatatctataactgtaactcctgggtcagggagtacaccaagtatcatgagcggctgctgactggcctcaaaggtagcaaaaggcttgttttcatagactgtcctgggaaactggctgagcacatagagcatgaacaacagaaactacctgctgccaccttggctttagaagaggacctgaaggtattccacaatgctctcaagctagctcacaaagacaccaaagtttctattaaagttggttctactgctgtccaagtaacttcagcagagcgaacaaaagtcctagggcaatcagtctttctaaatgacatttattatgcttcggaaattgaagaaatctgcctagtagatgagaaccagttcaccttaaccattgcaaaccagggcacgccgctcaccttcatgcaccaggagtgtgaagccattgtccagtctatcattcatatccggacccgctgggaactgtcacagcccgactctatcccccaacacaccaagattcggccaaaagatgtccctgggacactgctcaatatcgcattacttaatttaggcagttctgacccgagtttacggtcagctgcctataatcttctgtgtgccttaacttgtacctttaatttaaaaatcgagggccagttactagagacatcaggtttatgtatccctgccaacaacaccctctttattgtctctattagtaagacactggcagccaatgagccacacctcacgttagaatttttggaagagtgtatttctggatttagcaaatctagtattgaattgaaacacctttgtttggaatacatgactccatggctgtcaaatctagttcgtttttgcaagcataatgatgatgccaaacgacaaagagttactgctattcttgacaagctgataacaatgaccatcaatgaaaaacagatgtacccatctattcaagcaaaaatatggggaagccttgggcagattacagatctgcttgatgttgtactagacagtttcatcaaaaccagtgcaacaggtggcttgggatcaataaaagctgaggtgatggcagatactgctgtagctttggcttctggaaatgtgaaattggtttcaagcaaggttattggaaggatgtgcaaaataattgacaagacatgcttatctccaactcctactttagaacaacatcttatgtgggatgatattgctattttagcacgctacatgctgatgctgtccttcaacaattcccttgatgtggcagctcatcttccctacctcttccacgttgttactttcttagtagccacaggtccgctctcccttagagcttccacacatggactggtcattaatatcattcactctctgtgtacttgttcacagcttcattttagtgaagagaccaagcaagttttgagactcagtctgacagagttctcattacccaaattttacttgctgtttggcattagcaaagtcaagtcagctgctgtcattgccttccgttccagttaccgggacaggtcattctctcctggctcctatgagagagagacttttgctttgacatccttggaaacagtcacagaagctttgttggagatcatggaggcatgcatgagagatattccaacgtgcaagtggctggaccagtggacagaactagctcaaagatttgcattccaatataatccatccctgcaaccaagagctcttgttgtctttgggtgtattagcaaacgagtgtctcatgggcagataaagcagataatccgtattcttagcaaggcacttgagagttgcttaaaaggacctgacacttacaacagtcaagttctgatagaagctacagtaatagcactaaccaaattacagccacttcttaataaggactcgcctctgcacaaagccctcttttgggtagctgtggctgtgctgcagcttgatgaggtcaacttgtattcagcaggtaccgcacttcttgaacaaaacctgcatactttagatagtctccgtatattcaatgacaagagtccagaggaagtatttatggcaatccggaatcctctggagtggcactgcaagcaaatggatcattttgttggactcaatttcaactctaactttaactttgcattggttggacaccttttaaaagggtacaggcatccttcacctgctattgttgcaagaacagtcagaattttacatacactactaactctggttaacaaacacagaaattgtgacaaatttgaagtgaatacacagagcgtggcctacttagcagctttacttacagtgtctgaagaagttcgaagtcgctgcagcctaaaacatagaaagtcacttcttcttactgatatttcaatggaaaatgttcctatggatacatatcccattcatcatggtgacccttcctataggacactaaaggagactcagccatggtcctctcccaaaggttctgaaggataccttgcagccacctatccaactgtcggccagaccagtccccgagccaggaaatccatgagcctggacatggggcaaccttctcaggccaacactaagaagttgcttggaacaaggaaaagttttgatcacttgatatcagacacaaaggctcctaaaaggcaagaaatggaatcagggatcacaacaccccccaaaatgaggagagtagcagaaactgattatgaaatggaaactcagaggatttcctcatcacaacagcacccacatttacgtaaagtttcagtgtctgaatcaaatgttctcttggatgaagaagtacttactgatccgaagatccaggcgctgcttcttactgttctagctacactggtaaaatataccacagatgagtttgatcaacgaattctttatgaatacttagcagaggccagtgttgtgtttcccaaagtctttcctgttgtgcataatttgttggactctaagatcaacaccctgttatcattgtgccaagatccaaatttgttaaatccaatccatggaattgtgcagagtgtggtgtaccatgaagaatccccaccacaataccaaacatcttacctgcaaagttttggttttaatggcttgtggcggtttgcaggaccgttttcaaagcaaacacaaattccagactatgctgagcttattgttaagtttcttgatgccttgattgacacgtacctgcctggaattgatgaagaaaccagtgaagaatccctcctgactcccacatctccttaccctcctgcactgcagagccagcttagtatcactgccaaccttaacctttctaattccatgacctcacttgcaacttcccagcattccccaggaatcgacaaggagaacgttgaactctcccctaccactggccactgtaacagtggacgaactcgccacggatccgcaagccaagtgcagaagcaaagaagcgctggcagtttcaaacgtaatagcattaagaagatcgtgtgaagcttgcttgctttcttttttaaaatcaacttaacatgggctcttcactagtgaccccttccctgtccttgccctttccccccatgttgtaatgctgcacttcctgttttataatgaacccatccggtttgccatgttgccagatgatcaactcttcgaagccttgcctaaatttaatgctgccttttctttaactttttttcttctacttttggcgtgtatctggtatatgtaagtgttcagaacaactgcaaagaaagtgggaggtcaggaaacttttaactgagaaat

SEQ ID NO: 41 - DNA, 2257 bp, Homo sapiens
acggcagccgtcagggaccgtcccccaactcccctttccgctcaggcagggtcctcgcggcccatgctggccgctggggacccgcgcagcccagaccgttcccgggccggccagccggcaccatggtggccctgaggcctgtgcagcaactccaggggggctaaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggctttccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctaccgggaccgcgggatggaccagatatcaagagagccatccatagggagctggctgggggtttccgtgggagctccagaactttccccagctgagtgaagagcccagcccctcttatgtgcaattgccttgaactacgaccctgtagagatttctctcatggcgttctagttctctgacctgagtctttgttttaagaagtatttgtct

SEQ ID NO: 42 - DNA, 2969 bp, Homo sapiens
ccaggcagctggggtaaggagttcaaggcagcgcccacacccgggggctctccgcaacccgaccgcctgtccgctcccccacttcccgccctccctcccacctactcattcacccacccacccacccagagccgggacggcagcccaggcgcccgggccccgccgtctcctcgccgcgatcctggacttcctcttgctgcaggacccggcttccacgtgtgtcccggagccggcgtctcagcacacgctccgctccgggcctgggtgcctacagcagccagagcagcagggagtccgggacccgggcggcatctgggccaagttaggcgccgccgaggccagcgctgaacgtctccagggccggaggagccgcggggcgtccgggtctgagccgcagcaaatgggctccgacgtgcgggacctgaacgcgctgctgcccgccgtcccctccctgggtggcggcggcggctgtgccctgcctgtgagcggcgcggcgcagtgggcgccggtgctggactttgcgcccccgggcgcttcggcttacgggtcgttgggcggccccgcgccgccaccggctccgccgccacccccgccgccgccgcctcactccttcatcaaacaggagccgagctggggcggcgcggagccgcacgaggagcagtgcctgagcgccttcactgtccacttttccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttctcgttcagaccagctcaaaagacaccaaaggagacatacaggtgtgaaaccattccagtgtaaaacttgtcagcgaaagttctcccggtccgaccacctgaagacccacaccaggactcatacaggtgaaaagcccttcagctgtcggtggccaagttgtcagaaaaagtttgcccggtcagatgaattagtccgccatcacaacatgcatcagagaaacatgaccaaactccagctggcgctttgaggggtctccctcggggaccgttcagtgtcccaggcagcacagtgtgtgaactgctttcaagtctgactctccactcctcctcactaaaaaggaaacttcagttgatcttcttcatccaacttccaagacaagataccggtgcttctggaaactaccaggtgtgcctggaagagttggtctctgccctgcctacttttagttgactcacaggccctggagaagcagctaacaatgtctggttagttaaaagcccattgccatttggtgtggattttctactgtaagaagagccatagctgatcatgtccccctgacccttcccttctttttttatgctcgttttcgctggggatggaattattgtaccattttctatcatggaatatttataggccagggcatgtgtatgtgtctgctaatgtaaactttgtcatggtttccatttactaacagcaacagcaagaaataaatcagagagcaaggcatcgggggtgaatcttgtctaacattcccgaggtcagccaggctgctaacctggaaagcaggatgtagttctgccaggcaacttttaaagctcatgcatttcaagcagctgaagaaaaaatcagaactaaccagtacctctgtatagaaatctaaaagaattttaccattcagttaattcaatgtgaacactggcacactgctcttaagaaactatgaagatctgagatttttttgtgtatgtttttgactcttttgagtggtaatcatatgtgtctttatagatgtacatacctccttgcacaaatggaggggaattcattttcatcactgggagtgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaatccattgtttaaagatggtcgtgtgtgtgtgtgtgtgtgtgtgtgtgtgtgtgttgtgttgtgttttgttttttaagggagggaatttattatttaccgttgcttgaaattactgtgtaaatatatgtctgataatgatttgctctttgacaactaaaattaggactgtataagtactagatgcatcactgggtgttgatcttacaagatattgatgataacacttaaaattgtaacctgcatttttcactttgctctcaattaaagtctattcaaaag

SEQ ID NO: 43 - DNA, 69 bp, Artificial
  synthetic DNA
ggcctccataaagtaggaaacactacacagctccataaagtaggaaacactacattaattaagcggtac

SEQ ID NO: 44 - DNA, 61 bp, Artificial
  synthetic DNA
cgcttaattaatgtagtgtttcctactttatggagctgtgtagtgtttcctactttatgga

SEQ ID NO: 45 - DNA, 54 bp, Artificial
  synthetic DNA
tccataaagtaggaaacactacaggactccataaagtaggaaacactacagtac

SEQ ID NO: 46 - DNA, 52 bp, Artificial
  synthetic DNA
tgtagtgtttcctactttatggagtcctgtagtgtttcctactttatggaat

SEQ ID NO: 47 - DNA, 573 bp, Adenovirus
ttatggactggaataaaccctccacctaactgtcaaattgtggaaaacactaatacaaatgatggcaaacttactttagtattagtaaaaaacggagggcttgttaatggctacgtgtctctagttggtgtatcagacactgtgaaccaaatgttcacacaaaagacagcaaacatccaattaagattatattttgactcttctggaaatctattaactgatgaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaa

SEQ ID NO: 48 - DNA, 270 bp, Adenovirus
ggagttcttactcttaagtgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacacc

SEQ ID NO: 49 - DNA, 843 bp, Adenovirus
ggagttcttactcttaagtgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaacggagggcttgttaatggctacgtgtctctagttggtgtatcagacactgtgaaccaaatgttcacacaaaagacagcaaacatccaattaagattatattttgactcttctggaaatctattaactgatgaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaa

SEQ ID NO: 50 - DNA, 975 bp, Artificial
  synthetic DNA
atgaagcgcgcaagaccgtctgaagataccttcaaccccgtgtatccatatgacacggaaaccggtcctccaactgtgccttttcttactcctccctttgtatcccccaatgggtttcaagagagtccccctggagttcttactcttaagtgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaacggagggcttgttaatggctacgtgtctctagttggtgtatcagacactgtgaaccaaatgttcacacaaaagacagcaaacatccaattaagattatattttgactcttctggaaatctattaactgatgaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaa

(19) United States
(12) Patent Application Publication
(10) Pub. No.: US 2014/0199688 A1
(43) Pub. Date: Jul. 17, 2014

(54) CONDITIONALLLY REPLICATION-COMPETENT ADENOVIRUS

(71) Applicants: Hiroyuki Mizuguchi, Ibaraki-shi (JP); Fuminori Sakurai, Ibaraki-shi (JP)

(72) Inventors: Hiroyuki Mizuguchi, Ibaraki-shi (JP); Fuminori Sakurai, Ibaraki-shi (JP)

(73) Assignee: NATIONAL INSTITUTE OF BIOMEDICAL INNOVATION, Ibaraki-shi, Osaka (JP)

(21) Appl. No.: 14/240,216

(22) PCT Filed: Feb. 17, 2012
(86) PCT No.: PCT/JP2012/053814

§ 371 Date: Mar. 12, 2014

(30) Foreign Application Priority Data
Aug. 23, 2011 (JP) 2011-181414

(51) Int. Cl. C12Q 1/68; C12N 7/00; C12N 15/86

(52) U.S. Cl. CPC C12Q 1/6897; C12N 15/86; C12N 7/00
USPC 435/5; 435/320.1; 435/235.1

(57) ABSTRACT

The object of the present invention is to provide a novel conditionally replicating adenovirus and a reagent comprising the same for cancer cell detection or for cancer diagnosis.

The present invention provides a polynucleotide, which comprises human telomerase reverse transcriptase (hTERT) promoter, E1A gene, IRES sequence and E1B gene in this order and which comprises a target sequence of a first miRNA. The present invention also provides a recombinant adenovirus, which comprises a replication cassette comprising the above polynucleotide, wherein the replication cassette is integrated into the E1 region of the adenovirus genome.

### TECHNICAL FIELD

The present invention relates to a novel conditionally replicating adenovirus and a reagent comprising the same for cancer cell detection or for cancer diagnosis.

### BACKGROUND ART

Techniques currently used for cancer diagnosis mainly include (i) those using large-sized testing instruments (e.g., MRI) and (ii) those for measuring tumor markers or the like in blood, and expectations are now focused on (ii) which are simple techniques with less burden on patients. In particular, cancer cells circulating in the peripheral blood of cancer patients (i.e., circulating tumor cells (CTCs)) show a close relationship with clinical symptoms because these cells increase the risk of systemic metastasis and because the prognosis of patients with CTCs is significantly poor. Thus, it has been expected to develop a technique for simple and highly sensitive detection of CTCs as a predictive factor or surrogate marker for prognosis.

Techniques used for CTC detection include detection with a cancer-related antigen such as EpCAM (epithelial cell adhesion molecule) or cytokeratin-8 (e.g., CellSearch system) and detection by means of RT-PCR, etc. However, these cancer-related antigens are also expressed on normal epithelial cells and hence are highly likely to cause false positive detection, while cell morphology characteristic of cancer cells cannot be observed at the same time in the case of PCR detection. For these reasons, there has been a demand for a new technique in terms of sensitivity, simplicity, accuracy and costs.

On the other hand, the inventors of the present invention have already developed a conditionally replicating adenovirus which grows specifically in cancer cells and expresses GFP (GFP-expressing conditionally replicating adenovirus: GFP-CRAd) (which is referred to as TelomeScan®, OBP-401 or Telomelysin-GFP) (Patent Document 1: WO2006/036004). Moreover, the inventors of the present invention have also developed a simple technique for CTC detection using this TelomeScan (Non-patent Document 1: Kojima T., et al, J. Clin. Invest., 119; 3172, 2009).

However, since TelomeScan has the fiber protein of adenovirus type 5 and infects via coxsackievirus and adenovirus receptor (CAR) in target cells, TelomeScan may not infect cells which do not express CAR. In particular, it is known that CAR expression is reduced in highly malignant cancer cells which are highly invasive, metastatic and proliferative (Non-patent Document 2: Okegawa T., et al, Cancer Res., 61: 6592-6600, 2001); and hence TelomeScan may not detect these highly malignant cancer cells. Moreover, although less likely, TelomeScan may give false positive results by infecting and growing in normal blood cells (e.g., leukocytes) to cause GFP expression.

For these reasons, there has been a demand for a reagent for cancer cell detection and a reagent for cancer diagnosis, each of which detects almost all cancer cells including CAR-negative ones and does not give any false positive results in normal blood cells.

### PRIOR ART DOCUMENTS

### Patent Documents

Patent Document 1: WO2006/036004

Non-patent Document 1: Kojima T., et al, J. Clin. Invest., 119: 3172, 2009

Non-patent Document 2: Okegawa T., et al, Cancer Res., 61: 6592-6600, 2001

### SUMMARY OF THE INVENTION

### Problem to be Solved by the Invention

The present invention has been made under these circumstances, and the problem to be solved by the present invention is to provide a reagent for cancer cell detection and a reagent for cancer diagnosis, each of which detects almost all cancer cells including CAR-negative ones and does not give any false positive results in blood cells, as well as to provide a conditionally replicating recombinant adenovirus which is useful as such a reagent.

### Means to Solve the Problem

As a result of extensive and intensive efforts made to solve the above problem, the inventors of the present invention have found that not only CAR-positive cells, but also CAR-negative cells can be detected when the fiber of adenovirus type 5 in TelomeScan is replaced with another adenovirus fiber binding to CD46, which is highly expressed on almost all human cells, particularly cancer cells in general. Moreover, the inventors of the present invention have succeeded in avoiding any false positive results in blood cells by integration of a microRNA (miRNA)-mediated gene regulatory system into TelomeScan, which led to the completion of the present invention.

Namely, the present invention is as follows.

(1) A polynucleotide, which comprises human telomerase reverse transcriptase promoter, E1A gene, IRES sequence and E1B gene in this order and which comprises a target sequence of a first microRNA.

(2) The polynucleotide according to (1) above, wherein the first microRNA is expressed in non-cancer cells.

(3) The polynucleotide according to (1) or (2) above, wherein the first microRNA is at least one selected from the group consisting of miR-142, miR-15, miR-16, miR-21, miR-126, miR-181, miR-223, miR-296, miR-125, miR-143, miR-145, miR-199 and let-7.

(4) A recombinant adenovirus, which comprises a replication cassette comprising the polynucleotide according to any one of (1) to (3) above, wherein the replication cassette is integrated into the E1 region of the adenovirus genome.

(5) The recombinant adenovirus according to (4) above, which further comprises a labeling cassette comprising a reporter gene and a promoter capable of regulating the expression of the gene, wherein the labeling cassette is integrated into the E3 region of the adenovirus genome.

(6) The recombinant adenovirus according to (5) above, wherein the labeling cassette further comprises a target sequence of a second microRNA.

(7) The recombinant adenovirus according to (4) above, wherein a cell death-inducing cassette comprising a gene encoding a cell death induction-related protein and a promoter capable of regulating the expression of the gene is further integrated into the E3 region of the adenovirus genome.

(8) The recombinant adenovirus according to (7) above, wherein the cell death-inducing cassette further comprises a target sequence of a second microRNA.

(9) The recombinant adenovirus according to (6) or (8) above, wherein the second microRNA is expressed in non-cancer cells.

(10) The recombinant adenovirus according to (9) above, wherein the second microRNA is at least one selected from the group consisting of miR-142, miR-15, miR-16, miR-21, miR-126, miR-181, miR-223, miR-296, miR-125, miR-143, miR-145, miR-199 and let-7.

(11) The recombinant adenovirus according to (5) or (6) above, wherein the reporter gene is a gene encoding a protein which emits fluorescence or a gene encoding an enzyme protein which generates a luminophore or a chromophore upon enzymatic reaction.

(12) The recombinant adenovirus according to any one of (5) to (10) above, wherein the promoter is human telomerase reverse transcriptase promoter or cytomegalovirus promoter.

(13) The recombinant adenovirus according to any one of (4) to (12) above, which further comprises a gene encoding a CD46-binding fiber protein.

(14) The recombinant adenovirus according to (13) above, wherein the CD46-binding fiber protein comprises at least the fiber knob region in the fiber protein of adenovirus type 34 or 35.

(15) A reagent for cancer cell detection, which comprises the recombinant adenovirus according to any one of (4) to (14) above.

(16) A reagent for cancer diagnosis, which comprises the recombinant adenovirus according to any one of (4) to (14) above.

(17) The reagent according to (15) above, wherein the cancer cells are derived from a biological sample taken from a subject.

(18) The reagent according to (17) above, wherein the biological sample is blood.

(19) The reagent according to (15) or (18) above, wherein the cancer cells are circulating tumor cells.

(20) The reagent according to any one of (15) and (17) to (19) above, wherein the cancer cells are drug-resistant cancer cells.

(21) The reagent according to any one of (15) and (17) to (20) above, wherein the cancer cells are cancer stem cells.

(22) The reagent according to any one of (15) and (17) to (21) above, wherein the cancer cells are cancer cells having undergone epithelial-mesenchymal transition or mesenchymal-epithelial transition.

(23) A method for cancer cell detection, which comprises contacting cancer cells with the recombinant adenovirus according to (11) above and detecting the fluorescence or color produced by the cancer cells.

(24) The method according to (23) above, wherein the cancer cells are derived from a biological sample taken from a subject.

(25) The method according to (24) above, wherein the biological sample is blood.

(26) The method according to (25) above, wherein the cancer cells are circulating tumor cells.

### Effects of the Invention

The present invention enables simple and highly sensitive detection of CAR-negative cancer cells without detection of normal blood cells (e.g., leukocytes).

### BRIEF DESCRIPTION OF THE DRAWINGS

FIG. 1 is a schematic view showing an example of the structure of the recombinant adenovirus of the present invention.

FIG. 2 shows the results measured for activity of recombinant adenoviruses by flow cytometry.

FIG. 3 shows the results detected for H1299 cells contained in blood samples.

FIG. 4 shows the results detected for A549 cells contained in blood samples.

FIG. 5 shows the results measured for activity of the recombinant adenovirus of the present invention in various types of cancer cells.

FIG. 6 shows the results detected for cancer cells having undergone epithelial-mesenchymal transition (EMT).

FIG. 7 shows the results detected for cancer stem cells.

FIG. 8 shows the results detected for H1299 and T24 cells contained in blood samples by using a red fluorescent protein.

### DESCRIPTION OF EMBODIMENTS

The present invention will be described in more detail below. The following embodiments are illustrated to describe the present invention, and it is not intended to limit the present invention only to these embodiments. The present invention can be implemented in various modes, without departing from the spirit of the present invention. Moreover, this specification incorporates the contents disclosed in the specification and drawings of Japanese Patent Application No. 2011-181414 (filed on Aug. 23, 2011), based on which the present application claims priority.

1. Summary

TelomeScan (i.e., a conditionally replicating adenovirus comprising hTERT promoter, E1A gene, IRES sequence and E1B gene integrated in this order into the E1-deficient region of adenovirus type 5 and comprising cytomegalovirus (CMV) promoter and GFP integrated in this order into the E3-deficient region of adenovirus type 5), which has been previously developed by the inventors of the present invention, has problems in that: (i) TelomeScan may not detect highly malignant cancer cells where CAR expression is reduced; and (ii) TelomeScan may detect normal blood cells as false positive. As a result of extensive and intensive efforts made to solve these problems, the inventors of the present invention have found that highly malignant CAR-negative cancer cells can be detected when the fiber of adenovirus type 5 in TelomeScan is replaced with another adenovirus fiber binding to CD46, which is highly expressed on almost all human cells, particularly cancer cells in general. Moreover, the inventors of the present invention have also found that when a target sequence of miR-142-3p, which is miRNA, is integrated into each of the replication and labeling cassettes in TelomeScan, virus growth and labeling protein expression can be prevented in normal blood cells to thereby prevent the occurrence of false positive results in normal blood cells.

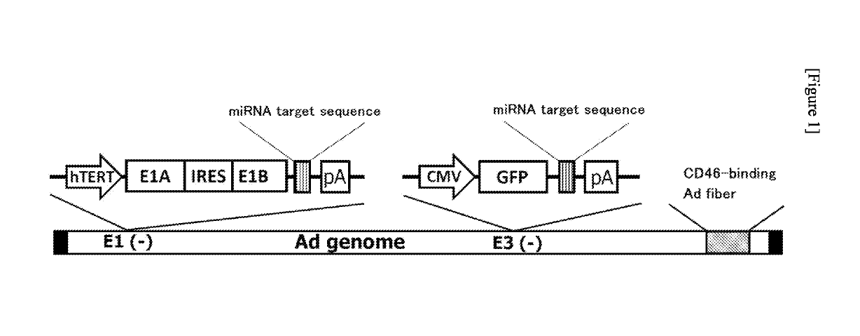

Namely, in a preferred embodiment of the present invention, the recombinant adenovirus of the present invention is a recombinant adenovirus, in which a replication cassette comprising hTERT promoter, E1A gene, IRES sequence, E1B gene and a target sequence of microRNA is integrated into the E1 region of the adenovirus genome and a labeling cassette comprising a reporter gene, a promoter capable of regulating the expression of the gene and a target sequence of microRNA is integrated into the E3 region of the adenovirus genome, and which comprises a gene encoding a CD46-binding adenovirus fiber protein (FIG. 1). This recombinant adenovirus has the following features.

(i) Because of comprising a gene encoding a CD46-binding adenovirus fiber protein, this recombinant adenovirus is able to infect almost all cells including CAR-negative cells.
(ii) Because of comprising hTERT promoter, this recombinant adenovirus grows specifically in hTERT-expressing cancer cells and also increases reporter gene expression upon growth, whereby the production of a labeling protein, a chromophore or the like can be increased to detectable levels.
(iii) Because of comprising a target sequence of miRNA, this recombinant adenovirus can prevent the occurrence of false positive results even when the virus infects normal cells having hTERT promoter activity, because expression of this miRNA prevents not only growth of the virus but also expression of the reporter gene. In particular, because of comprising a target sequence of miRNA which is expressed specifically in blood cells, this recombinant adenovirus can prevent the occurrence of false positive results even when the virus infects normal blood cells having hTERT promoter activity, because expression of this miRNA prevents not only growth of the virus in blood cells but also expression of the reporter gene.

The present invention has been completed on the basis of these findings.

2. Recombinant Adenovirus

### (1) Replication Cassette

The present invention relates to a polynucleotide, which comprises human telomerase reverse transcriptase (hTERT) promoter, E1A gene, IRES sequence and E1B gene in this order and which comprises a target sequence of microRNA. In addition, the present invention relates to a recombinant adenovirus, which comprises a replication cassette comprising the above polynucleotide, wherein the replication cassette is integrated into the E1 region of the adenovirus genome.

By the action of the above polynucleotide (or a replication cassette comprising the same), the recombinant adenovirus of the present invention can grow specifically in cancer cells and can also be prevented from growing in cells which express the desired miRNA. For example, if the target sequence of miRNA contained in the replication cassette of the present invention is a target sequence of miRNA which is expressed specifically in blood cells, the recombinant adenovirus of the present invention grows specifically in hTERT-expressing cancer cells and is prevented from growing in blood cells.

Human telomerase reverse transcriptase (hTERT) promoter is a promoter for reverse transcriptase which is an element of human telomerase. Although human telomerase activity will be increased by splicing of hTERT mRNA, post-translational modification of hTERT protein and other events, enhanced hTERT gene expression, i.e., increased hTERT promoter activity is thought to be the most important molecular mechanism. Human telomerase has been confirmed to show increased activity in 85% or more of human cancers, whereas it shows no activity in most normal cells. Thus, the use of hTERT promoter allows a gene downstream thereof to be expressed specifically in cancer cells. In the present invention, the hTERT promoter is located upstream of E1A gene, IRES sequence and E1B gene, whereby the virus can grow specifically in hTERT-expressing cancer cells.

hTERT has been confirmed to have many transcription factor binding sequences in a 1.4 kbp region upstream of its 5′-terminal end, and this region is regarded as hTERT promoter. In particular, a 181 bp sequence upstream of the translation initiation site is a core region important for expression of its downstream genes. In the present invention, although any sequence may be used as long as it includes this core region, an upstream sequence of approximately 378 bp which covers this core region in its entirety is preferred for use as the hTERT promoter. This sequence of approximately 378 bp has been confirmed to have the same efficiency of gene expression as the 181 bp core region alone. The nucleotide sequence of a 455 bp long hTERT promoter is shown in SEQ ID NO: 1.

In addition to the sequence shown in SEQ ID NO: 1, the nucleotide sequence of hTERT promoter includes the nucleotide sequences of polynucleotides which are hybridizable under stringent conditions with DNA consisting of a nucleotide sequence complementary to DNA consisting of SEQ ID NO: 1 and which have hTERT promoter activity. Such polynucleotides may be obtained from cDNA and genomic libraries by known hybridization techniques (e.g., colony hybridization, plaque hybridization, Southern blotting) using a polynucleotide which consists of the nucleotide sequence shown in SEQ ID NO: 1 or a fragment thereof as a probe.

For preparation of cDNA libraries, reference may be made to “Molecular Cloning, A Laboratory Manual 2nd ed.” (Cold Spring Harbor Press (1989)). Alternatively, commercially available cDNA and genomic libraries may also be used for this purpose.

Stringent conditions in the above hybridization include, for example, conditions of 1×SSC to 2×SSC, 0.1% to 0.5% SDS and 42° C. to 68° C., more specifically prehybridization at 60° C. to 68° C. for 30 minutes or longer and the subsequent 4 to 6 washings in 2×SSC, 0.1% SDS at room temperature for 5 to 15 minutes.

As to detailed procedures for hybridization, reference may be made to “Molecular Cloning, A Laboratory Manual 2nd ed.” (Cold Spring Harbor Press (1989); particularly Section 9.47-9.58), etc.

E1A and E1B genes are both included in the E1 gene of adenovirus. This E1 gene refers to one of the early genes among the virus early (E) and late (L) genes related to DNA replication, and it encodes a protein related to the regulation of viral genome transcription. EIA protein encoded by the E1A gene of adenovirus activates the transcription of a group of genes (e.g., E1B, E2, E4) required for infectious virus production. E1B protein encoded by the E1B gene of adenovirus assists late gene (L gene) mRNAs to accumulate into the cytoplasm of infected host cells and inhibits protein synthesis in the host cells, thereby facilitating virus replication. The nucleotide sequences of the E1A and E1B genes are shown in SEQ ID NO: 2 and SEQ ID NO: 3, respectively. In addition to the sequences shown in SEQ ID NO: 2 and SEQ ID NO: 3, the nucleotide sequences of the E1A and E1B genes include nucleotide sequences which are hybridizable under stringent conditions with DNA consisting of a nucleotide sequence complementary to DNA consisting of SEQ ID NO: 2 or SEQ ID NO: 3 and which encode a protein having E1A or E1B activity. Procedures and stringent conditions for hybridization are the same as those described above for the hTERT promoter.

IRES (internal ribosome entry site) sequence is a protein synthesis initiation signal specific to the picornavirus family and is considered to serve as a ribosomal binding site because of having a sequence complementary to the 3′-terminal end of 18S ribosomal RNA. It is known that translation of mRNAs derived from viruses of the picornavirus family is mediated by this sequence. The efficiency of translation from the IRES sequence is high and protein synthesis occurs even from the middle of mRNA in a manner not dependent on the cap structure. Thus, in the virus of the present invention, the E1A gene and the E1B gene, which is located downstream of the IRES sequence, are both translated independently by the action of hTERT promoter. With the use of the IRES sequence, hTERT promoter-mediated expression regulation occurs independently in both the E1A gene and the E1B gene, and hence virus growth can be more strictly limited to cells having telomerase activity when compared to the case where any one of the E1A gene or the E1B gene is regulated by the hTERT promoter. Moreover, the IRES sequence inserted between the E1A gene and the E1B gene can increase the growth capacity of the virus in host cells. The nucleotide sequence of the IRES sequence is shown in SEQ ID NO: 4. In addition to the sequence shown in SEQ ID NO: 4, the nucleotide sequence of the IRES sequence includes nucleotide sequences which are hybridizable under stringent conditions with DNA consisting of a nucleotide sequence complementary to DNA consisting of SEQ ID NO: 4 and which encode a protein having IRES activity. Procedures and stringent conditions for hybridization are the same as those described above for the hTERT promoter.

miRNA generally refers to short single-stranded RNA of approximately 15 to 25 nucleotides and is considered to regulate the translation of various genes upon binding to its target sequence present in mRNA. Thus, for example, when miRNA-expressing cells are infected with a recombinant adenovirus comprising a desired gene and a target sequence of the miRNA, the desired gene is prevented from being expressed in these cells. Such a target sequence of miRNA may be inserted into any site as long as a desired gene is prevented from being expressed, but it preferably inserted into an untranslated region of the desired gene, more preferably downstream of the desired gene.

The target sequence of miRNA to be used in the present invention includes target sequences of miRNAs which are expressed in non-cancer cells. Non-cancer cells are intended to mean cells that are not malignant tumor cells, and examples include normal cells, benign tumor cells and so on. Normal cells include, for example, normal blood cells, normal endothelial cells, normal fibroblasts, normal stem cells and so on. On the other hand, circulating tumor cells are regarded as cells originating from malignant tumors, and hence they fall within malignant tumor cells in the present invention.

The target sequence of miRNA to be used in the present invention also includes target sequences of miRNAs which are expressed specifically in blood cells. In the present invention, “blood cells” may include not only normal blood cells, but also cancerous blood cells. Namely, in the present invention, “miRNA which is expressed specifically in blood cells” may be expressed specifically in normal blood cells or may be expressed specifically in both normal blood cells and cancerous blood cells. Even when expressed specifically in both normal blood cells and cancerous blood cells, miRNA can also reduce false positive cases of normal blood cells during detection of circulating tumor cells and thereby ensures accurate detection of circulating tumor cells released from solid cancers. In the present invention, “miRNA which is expressed specifically in blood cells” is more preferably miRNA which is expressed in normal blood cells but is not expressed in cancerous blood cells.

In the present invention, blood cells include, but are not limited to, leukocytes (i.e., neutrophils, eosinophils, basophils, lymphocytes (T cells and B cells), monocytes, dendritic cells), CD34-positive cells, hematopoietic cells, hematopoietic stem cells, hematopoietic progenitor cells, peripheral blood mononuclear cells (PBMCs) and so on. Likewise, cancerous blood cells include leukemia cells, lymphoma cells and so on. In the present invention, being “expressed specifically” in certain cells is intended to mean not only that expression is limited only to the intended cells, but also that expression levels are higher in the intended cells than in other cells. For example, being “expressed specifically in blood cells” is intended to mean not only that expression is limited only to blood cells, but also that expression levels are higher in blood cells than in any cells other than blood cells.

miRNA which is expressed specifically in blood cells includes, for example, miR-142, miR-15, miR-16, miR-21, miR-126, miR-181, miR-223, miR-296 and so on, with miR-142, miR-15 and miR-16 being preferred.

Although miRNA is single-stranded RNA, it is possible to use a target sequence of either strand of premature double-stranded RNA as long as a desired gene can be prevented from being expressed. For example, there are miR-142-3p and miR-142-5p for miR-142, and a target sequence of either miRNA may be used in the present invention. Namely, in the present invention, “miR-142” includes both miR-142-3p and miR-142-5p, with miR-142-3p being preferred. Likewise, in the present invention, “miR-15” includes the sense strand (referred to as “miR-15S”) and antisense strand (referred to as “miR-15AS”) of premature double-stranded RNA. The same applies to other miRNAs.

miR-142-3p gene is located at a site where translocation occurs in B cell leukemia (aggressive B cell leukemia), and is known to be expressed in hematopoietic tissues (e.g., bone marrow, spleen, thymus), but not expressed in other tissues. Moreover, miR-142-3p has been observed to be expressed in mouse fetal liver (fetal hematopoietic tissue) and hence is considered to be involved in differentiation of the hematopoietic system (Chang-Zheng Chen, et al., Science, 2004).

In this embodiment, gene expression is regulated in two stages in a selective manner, because specific gene expression is caused in cancer cells by the action of hTERT promoter and gene expression in blood cells is regulated by the action of miRNA.

In another embodiment, the target sequence of miRNA to be used in the present invention includes a target sequence of miRNA whose expression is suppressed in cancer cells. miRNA whose expression is suppressed in cancer cells includes, for example, miR-125, miR-143, miR-145, miR-199, let-7 and so on. In this embodiment, specific gene expression in cancer cells is doubly regulated by the action of hTERT promoter and miRNA.

Although miRNA molecules have been initially found in nematodes, yeast and other organisms, there are currently found several hundreds of miRNAs in humans and mice. The sequences of these miRNAs are known, and sequence information and so on can be obtained by access to public DBs (e.g., miRBase sequence database (http://microrna.sanger.ac.uk/sequences/index.shtml, http://www.mirbase.org/)).

The sequences of miR-142, miRNA-15, miRNA-16, miR-21, miR-126, miR-181, miR-223, miR-296, miR-125, miR-143, miR-145, miR-199 and let-7 are shown below.

| (SEQ ID NO: 5) | |
|---|---|
| miR-142-3p: | 5′-UGUAGUGUUUCCUACUUUAUGGA |
| (SEQ ID NO: 6) | |
| miR-142-5p: | 5′-CAUAAAGUAGAAAGCACUACU |
| (SEQ ID NO: 7) | |
| miR-15S: | 5′-UAGCAGCACAUAAUGGUUUGUG |
| (SEQ ID NO: 8) | |
| miR-15AS: | 5′-CAGGCCAUAUUGUGCUGCCUCA |
| (SEQ ID NO: 9) | |
| miR-16S: | 5′-UAGCAGCACGUAAAUAUUGGCG |
| (SEQ ID NO: 10) | |
| miR-16AS: | 5′-CCAGUAUUAACUGUGCUGCUGA |
| (SEQ ID NO: 11) | |
| miR-21S: | 5′-UAGCUUAUCAGACUGAUGUUGA |
| (SEQ ID NO: 12) | |
| miR-21AS: | 5′-CAACACCAGUCGAUGGGCUGU |
| (SEQ ID NO: 13) | |
| miR-126S: | 5′-UCGUACCGUGAGUAAUAAUGCG |
| (SEQ ID NO: 14) | |
| miR-126AS: | 5′-CAUUAUUACUUUUGGUACGCG |
| (SEQ ID NO: 15) | |
| miR-181: | 5′-AACAUUCAACGCUGUCGGUGAGU |
| (SEQ ID NO: 16) | |
| miR-223S: | 5′-UGUCAGUUUGUCAAAUACCCCA |
| (SEQ ID NO: 17) | |
| miR-223AS: | 5′-CGUGUAUUUGACAAGCUGAGUU |
| (SEQ ID NO: 18) | |
| miR-296-3p: | 5′-GAGGGUUGGGUGGAGGCUCUCC |
| (SEQ ID NO: 19) | |
| miR-296-5p: | 5′-AGGGCCCCCCCUCAAUCCUGU |
| (SEQ ID NO: 20) | |
| miR-125: | 5′-UCCCUGAGACCCUUUAACCUGUGA |
| (SEQ ID NO: 21) | |
| miR-143S: | 5′-UGAGAUGAAGCACUGUAGCUC |
| (SEQ ID NO: 22) | |
| miR-143AS: | 5′-GGUGCAGUGCUGCAUCUCUGGU |
| (SEQ ID NO: 23) | |
| miR-145S: | 5′-GUCCAGUUUUCCCAGGAAUCCCU |
| (SEQ ID NO: 24) | |
| miR-145AS: | 5′-GGAUUCCUGGAAAUACUGUUCU |
| (SEQ ID NO: 25) | |
| miR-199: | 5′-CCCAGUGUUCAGACUACCUGUUC |
| (SEQ ID NO: 26) | |
| let-7: | 5′-UGAGGUAGUAGGUUGUAUAGUU |

In the present invention, a single unit of a target sequence of miRNA is composed of a sequence complementary to the whole or part of the miRNA, and has a nucleotide length of 7 to 30 nucleotides, preferably 19 to 25 nucleotides, more preferably 21 to 23 nucleotides. In the present invention, a single unit of a target sequence of miRNA is intended to mean a nucleotide sequence having the minimum length required for serving as a target of certain miRNA. More specifically, it is intended to mean an oligonucleotide of at least 7 nucleotides in length selected from complementary sequences of the nucleotide sequences shown in SEQ ID NOs: 5 to 26, and such an oligonucleotide may comprise substitution, deletion, addition or removal of one or several nucleotides at any site(s).

The target sequence as a whole to be integrated into the polynucleotide or recombinant adenovirus of the present invention may comprise several copies of a single unit of target sequence in order to ensure effective interaction between miRNA and the target sequence. The target sequence as a whole to be integrated into the recombinant adenovirus may be of any length as long as it can be integrated into the viral genome. For example, it may comprise 1 to 10 copies, preferably 2 to 6 copies, and more preferably 2 or 4 copies of a single unit of target sequence (John G. Doench, et al., Genes Dev. 2003 17:438-442). An oligonucleotide of appropriate length may be inserted between single units of target sequence contained in the target sequence as a whole. The length of such an oligonucleotide of appropriate length is not limited in any way as long as the target sequence as a whole can be integrated into the recombinant adenovirus genome. For example, such an oligonucleotide may be of 0 to 8 nucleotides in length. Moreover, in the case of comprising several units of a target sequence of miRNA, the target sequences in the respective units may be those toward the same miRNA or those toward different miRNAs. Furthermore, in the case of comprising target sequences toward the same miRNA, the target sequences in the respective units may have different lengths and/or different nucleotide sequences.

The target sequence of miRNA to be contained in the polynucleotide of the present invention (or a replication cassette comprising the same) can also be referred to as a “target sequence of a first microRNA” in order that the polynucleotide, when integrated into the recombinant adenovirus, should be distinguished from other miRNA target sequences present in the recombinant adenovirus.

When miR-142-3p is used as miRNA in the present invention, a target sequence thereof may be exemplified by sequences comprising the following sequences, by way of example.

(i) Sequence comprising two units of a target sequence of miR-142-3p:
(SEQ ID NO: 27, each underline represents a single unit of a target
sequence of miR-142-3p)
5′-gcggcctccataaagtaggaaacactacacagctccataaagtaggaaacactacattataagcggtac
(ii) Sequence comprising four units of a target sequence of miR-142-3p:
(SEQ ID NO: 28, each underline represents a single unit of a target
sequence of miR-142-3p)
5′-ggcctccataaagtaggaaacactacacagctccataaagtaggaaacactacattaattccataaagtaggaaacactac
accactccataaagtaggaaacactacagtac

In the present invention, a target sequence of miRNA is placed downstream of the construct of hTERT promoter-E1A gene-IRES sequence-E1B gene, and the resulting polynucleotide comprising the hTERT promoter, the E1A gene, the IRES sequence, the E1B gene and the target sequence of miRNA in this order (which polynucleotide is referred to as a replication cassette) is integrated into the adenovirus genome, whereby E1 gene expression and virus growth can be prevented in cells expressing the miRNA.

In the present invention, a target sequence of miRNA is integrated downstream of the E1B gene or the reporter gene described later, whereby a gene located upstream thereof is prevented from being expressed. Although the details of this mechanism are not clear, a possible mechanism is as follows. First, miRNA-RISC (RNA-induced silencing complex) cleaves a target sequence on mRNA to thereby remove polyA from the mRNA. This would reduce the stability of the mRNA to cause degradation of the mRNA and hence prevention of gene expression. Alternatively, miRNA-RISC would recruit polyA ribonuclease, as in the case of normal miRNA, to cause polyA degradation, as a result of which the stability of mRNA would be reduced and gene expression would be prevented.

It should be noted that there are previous reports showing that the miRNA-induced inhibitory effect against gene expression was not obtained for the expression (translation) of a gene inserted downstream of the IRES sequence (Ramesh S. Pillai et al., Science 309, 1573(2005); Geraldine Mathonnet, et al., Science 317, 1764 (2007)). However, when the inventors of the present invention confirmed gene expression for the recombinant adenovirus of the present invention comprising hTERT promoter, E1A gene, IRES sequence, E1B gene and a target sequence of miRNA in this order, the miRNA was found to sufficiently prevent the expression of the E1B gene inserted downstream of the IRES sequence. This is a new finding in the present invention.

The genes to be contained in the replication cassette of the present invention can be obtained by standard genetic engineering techniques. For example, it is possible to use nucleic acid synthesis with a DNA synthesizer, which is commonly used as a genetic engineering technique. Alternatively, it is also possible to use PCR techniques in which gene sequences serving as templates are isolated or synthesized, and primers specific to each gene are then designed to amplify the gene sequence with a PCR system (Current Protocols in Molecular Biology, John Wiley & Sons (1987) Section 6.1-6.4) or gene amplification techniques using a cloning vector. The above techniques can be easily accomplished by those skilled in the art in accordance with Molecular cloning 2^{nd} Edt. Cold Spring Harbor Laboratory Press (1989), etc. For purification of the resulting PCR product, known techniques can be used. If necessary, conventionally used sequencing techniques may be used to confirm whether the intended gene has been obtained, as expected. For example, dideoxynucleotide chain termination sequencing (Sanger et al. (1977) Proc. Natl. Acad. Sci. USA 74: 5463) or the like may be used for this purpose. Alternatively, an appropriate DNA sequencer (e.g., ABI PRISM (Applied Biosystems)) may also be used for sequence analysis.

In the present invention, the target sequence of miRNA can be obtained by being designed and synthesized such that each single unit of target sequence is complementary to the whole or part of the nucleotide sequence of the miRNA. For example, a target sequence of miR-142-3p can be obtained by synthesizing DNA such that it is complementary to the nucleotide sequence of miR-142-3p.

Then, the respective genes obtained as above are ligated in a given order. First, the above genes are each cleaved with known restriction enzymes or the like, and the cleaved DNA fragment of each gene is inserted into and ligated to a known vector in accordance with known procedures. As a known vector, pIRES vector may be used, by way of example. The pIRES vector comprises the IRES (internal ribosome entry site) sequence of encephalomyocarditis virus (ECMV) and is capable of translating two open reading frames (ORFs) from one mRNA. With the use of the pIRES vector, it is possible to prepare a “polynucleotide which comprises hTERT promoter, E1A gene, IRES sequence and E1B gene in this order and which comprises a target sequence of microRNA” by sequentially inserting the required genes into a multicloning site. Such a target sequence of miRNA may be inserted into any site, but it is preferably inserted downstream of the hTERT promoter-EIA-IRES-E1B construct. For DNA ligation, DNA ligase may be used. Alternatively, CMV promoter contained in a known vector (e.g., pShuttle) may be removed with known restriction enzymes and a sequence cleaved from the hTERT promoter-EIA-IRES-E1B-miRNA target sequence with appropriate restriction enzymes may then be inserted into this site, if necessary. Once the E1 gene required for adenovirus growth is allowed to be expressed under the control of the hTERT promoter, the virus can be grown specifically in cancer cells.

(2) Labeling Cassette

In yet another embodiment, the present invention relates to a recombinant adenovirus in which the above replication cassette is integrated into the E1 region of the adenovirus genome and a labeling cassette is further integrated into the E3 region of the adenovirus genome. Such a labeling cassette comprises a reporter gene and a promoter capable of regulating the expression of the gene, and may further comprise a target sequence of miRNA.

The adenovirus E3 region contains 11.6 kDa ADP (adenovirus death protein), and ADP has the function of promoting cell damage and virus diffusion. The recombinant adenovirus of the present invention is designed to eliminate any viral genome region like the E3 region containing ADP, which encodes a protein having the function of promoting cell damage and virus diffusion, so that the timing of cell death is delayed to facilitate identification of cancer tissues by production (emission, expression) of fluorescence (e.g., GFP). This is also effective in that circulating tumor cells (CTCs) described later can be detected alive over a long period of time.

The reporter gene to be contained in the labeling cassette in the recombinant adenovirus of the present invention is not limited in any way, and examples include a gene encoding a protein which emits fluorescence, a gene encoding an enzyme protein which generates a luminophore or a chromophore upon enzymatic reaction, a gene encoding an antibiotic, a gene encoding a tag-fused protein, a gene encoding a protein which is expressed on the cell surface and binds to a specific antibody, a gene encoding a membrane transport protein, and so on. Examples of a protein which emits fluorescence (i.e., a labeling protein) include a green fluorescent protein (GFP) derived from luminous jellyfish such as Aequorea victorea, its variants EGFP (enhanced-humanized GFP) and rsGFP (red-shift GFP), a yellow fluorescent protein (YFP), a cyan fluorescent protein (CFP), a blue fluorescent protein (BFP), GFP derived from Renilla reniformis and so on, and genes encoding these proteins can be used in the present invention. The above protein which emits fluorescence is preferably GFP or EGFP.

Likewise, examples of an enzyme protein which generates a luminophore or a chromophore upon enzymatic reaction include 3-galactosidase, luciferase and so on. β-Galactosidase generates a blue chromophore from 5-bromo-4-chloro-3-indolyl-3-D-galactopyranoside (X-gal) upon enzymatic reaction. On the other hand, luciferase generates a luminophore upon enzymatic reaction with luciferin. Firefly luciferase, bacterial luciferase, Renilla luciferase and so on are known as members of luciferase, and those skilled in the art would be able to select an appropriate enzyme from known luciferase members.

Moreover, the promoter capable of regulating the expression of the above gene is not limited in any way as long as it is a suitable promoter compatible with the virus used for the expression of the above desired gene. Examples include, but are not limited to, CMV promoter, hTERT promoter, SV40 late promoter, MMTV LTR promoter, RSV LTR promoter, SRα promoter, β-actin promoter, PGK promoter, EF-1a promoter and so on. Preferably, CMV promoter or hTERT promoter can be used for this purpose.

The target sequence of miRNA to be integrated into the labeling cassette may be either the same or different from the target sequence of miRNA to be integrated into the replication cassette.

In the present invention, the target sequence of miRNA is placed within the untranslated region of the reporter gene, preferably downstream of this gene, whereby the reporter gene can be prevented from being expressed. Namely, in the present invention, the labeling cassette preferably comprises a promoter capable of regulating the reporter gene, the reporter gene and the target sequence of microRNA in this order. The target sequence of miRNA to be integrated into the labeling cassette is referred to as a “target sequence of a second microRNA” in order that it should be distinguished from the target sequence of miRNA to be contained in the replication cassette. Other explanations on miRNA are the same as described above.

Details on how to obtain, purify and sequence the recombinant genes to be contained in the labeling cassette of the present invention are the same as described above for the replication cassette.

(3) Cell Death-Inducing Cassette

In yet another embodiment, the present invention relates to a recombinant adenovirus in which the above replication cassette is integrated into the E1 region of the adenovirus genome and a cell death-inducing cassette is integrated into the E3 region of the adenovirus genome. Such a cell death-inducing cassette comprises a gene encoding a cell death induction-related protein and a promoter capable of regulating the expression of the gene, and may further comprise a target sequence of microRNA.

The cell death-inducing cassette used in the recombinant adenovirus of the present invention comprises a gene encoding a cell death induction-related protein and a promoter capable of regulating the expression of the gene. Thus, for example, when the recombinant adenovirus of the present invention is infected into cancer cells, the virus grows specifically in the cancer cells to thereby increase the intracellular expression level of the cell death induction-related protein and induce cell death only in the cancer cells without damaging other normal cells.

Such a gene encoding a cell death induction-related protein is intended to mean a gene encoding a protein related to the induction of cell death in specific cells. Examples of a cell death induction-related protein include immunological proteins such as PA28. PA28 is a protein which activates intracellular proteasomes and which elicits immune reactions and also induces cell death when overexpressed. Moreover, TRAIL can also be exemplified as an apoptosis-inducing protein. TRAIL refers to a molecule which induces apoptotic cell death upon binding to its receptor on the cell surface.

Moreover, another example of the gene encoding a cell death induction-related protein is a tumor suppressor gene, which has the function of suppressing the growth of cancer cells. Examples of such a tumor suppressor gene include the following genes used in conventional gene therapy. SEQ ID NO (nucleotide sequence) and GenBank Accession No. are shown below for each gene.
p53 (SEQ ID NO: 29; Accession No. M14694): multiple types of cancer
p15 (SEQ ID NO: 30; Accession No. L36844): multiple types of cancer
p16 (SEQ ID NO: 31; Accession No. L27211): multiple types of cancer
APC (SEQ ID NO: 32; Accession No. M74088): colorectal cancer, gastric cancer, pancreatic cancer
BRCA-1 (SEQ ID NO: 33; Accession No. U14680): ovarian cancer, breast cancer
DPC-4 (SEQ ID NO: 34; Accession No. U44378): colorectal cancer, pancreatic cancer
FHIT (SEQ ID NO: 35; Accession No. NM 112012): gastric cancer, lung cancer, uterine cancer
p73 (SEQ ID NO: 36; Accession No. Y11416): neuroblastoma
PATCHED (SEQ ID NO: 37; Accession No. U59464): basal cell carcinoma
Rbp110 (SEQ ID NO: 38; Accession No. M15400): lung cancer, osteosarcoma
DCC (SEQ ID NO: 39; Accession No. X76132): colorectal cancer
NF1 (SEQ ID NO: 40; Accession No. NM 000267): neurofibroma type 1
NF2 (SEQ ID NO: 41; Accession No. L11353): neurofibroma type 2
WT-1 (SEQ ID NO: 42; Accession No. NM 000378): Wilms tumor

The target sequence of miRNA to be contained in the cell death-inducing cassette may be either the same or different from the target sequence of miRNA to be integrated into the replication cassette. In the present invention, the target sequence of miRNA is placed within the untranslated region of the gene encoding a cell death induction-related protein, preferably downstream of this gene, whereby the cell death induction-related protein can be prevented from being expressed. Namely, in the present invention, the cell death-inducing cassette preferably comprises a promoter capable of regulating the gene encoding a cell death induction-related protein, the gene encoding a cell death induction-related protein and the target sequence of microRNA in this order. Other explanations on miRNA are the same as described above.

Details on how to obtain, purify and sequence the recombinant genes to be contained in the cell death-inducing cassette of the present invention are the same as described above for the replication cassette.

To determine whether or not cell death has been induced, morphological observation described below may be conducted for this purpose. Namely, once cells adhered onto the bottom surface of a culture vessel have been infected with the recombinant virus of the present invention and incubated for a given period, the cells will be rounded and detached from the bottom surface and then will float as shiny cells in the culture solution, as observed under an inverted microscope. At this stage, the cells have lost their vital mechanism and hence a determination can be made that cell death has been induced. Alternatively, cell death can also be confirmed with a commercially available kit for living cell assay which uses a tetrazolium salt (e.g., MTT, XTT).

(4) CD46-Binding Fiber Protein

In yet another embodiment, the recombinant adenovirus of the present invention may comprise a gene encoding a CD46-binding adenovirus fiber protein.

Adenovirus vectors which are now commonly used are prepared structurally based on adenovirus type 5 (or type 2) belonging to Subgroup C among 51 serotypes of human adenovirus. Although adenovirus type 5 is widely used because of its excellent gene transfer properties, adenovirus of this type has a problem of being difficult to infect cells with low expression of coxsackievirus and adenovirus receptor (CAR) because its infection is mediated by binding to CAR on target cells. In particular, CAR expression is reduced in highly malignant cancer cells which are highly invasive, metastatic and proliferative, and hence an adenovirus having the fiber protein of adenovirus type 5 may not infect such highly malignant cancer cells.

In contrast, CD46 is expressed on almost all cells except for erythrocytes in humans and is also expressed on highly malignant cancer cells. Thus, a recombinant adenovirus comprising a gene encoding a CD46-binding adenovirus fiber protein can also infect CAR-negative and highly malignant cancer cells. For example, adenovirus types 34 and 35 bind to CD46 as their receptor and thereby infect cells (Marko Marttila, et al., J. Virol. 2005, 79(22):14429-36). As described above, CD46 is expressed on almost all cells except for erythrocytes in humans, and hence adenovirus types 34 and 35 are able to infect a wide range of cells including CAR-negative cells. Moreover, the fiber of adenovirus consists of a knob region, a shaft region and a tail region, and adenovirus infects cells through binding of its fiber knob region to the receptor. Thus, at least the fiber knob region in the fiber protein is replaced from adenovirus type 5 origin to adenovirus type 34 or 35 origin, whereby the virus will be able to infect CAR-negative cells via CD46.

Because of comprising a gene encoding a CD46-binding adenovirus fiber protein, the recombinant adenovirus of the present invention is able to infect almost all cells except for erythrocytes and thus able to infect highly malignant CAR-negative cancer cells which are highly invasive, metastatic and proliferative. In the present invention, “CAR-negative” cells are intended to mean cells where CAR expression is low or cells where CAR is not expressed at all.

57 serotypes have now been identified for human adenovirus, and these serotypes are classified into six groups, i.e., Groups A to F. Among them, adenovirus types belonging to Group B have been reported to bind to CD46. Adenovirus types belonging to Group B include adenovirus types 34 and 35, as well as adenovirus types 3, 7, 11, 16, 21 and 50, by way of example.

For use as a CD46-binding adenovirus fiber protein in the present invention, preferred is the fiber protein of adenovirus belonging to Group B, more preferred is the fiber protein of adenovirus type 3, 7, 34, 35, 11, 16, 21 or 50, and even more preferred is the fiber protein of adenovirus type 34 or 35.

The nucleotide sequence of a gene encoding the fiber protein of adenovirus type 34, 35, 3, 7, 11, 16, 21 or 50 is available from a known gene information database, e.g., the GenBank of NCBI (The National Center for Biotechnology Information). Moreover, in the present invention, the nucleotide sequence of a gene encoding the fiber protein of adenovirus type 34, 35, 3, 7, 11, 16, 21 or 50 includes not only the nucleotide sequence of each gene available from a database as described above, but also nucleotide sequences which are hybridizable under stringent conditions with DNA consisting of a nucleotide sequence complementary to DNA consisting of each nucleotide sequence available from a database and which encode a protein with binding activity to CD46.

The binding activity to CD46 can be evaluated when a recombinant adenovirus having DNA comprising the nucleotide sequence is measured for its infectivity to CD46-expressing cells. The infectivity of such a recombinant adenovirus may be measured in a known manner, for example, by detecting GFP expressed by the virus infected into CD46-expressing cells under a fluorescence microscope or by flow cytometry, etc. Procedures and stringent conditions for hybridization are the same as described above.

The recombinant adenovirus of the present invention may comprise the entire or partial region of a CD46-binding adenovirus fiber protein, such that at least the fiber knob region in the fiber protein binds to CD46. Namely, in the present invention, the CD46-binding adenovirus fiber protein may comprise at least the fiber knob region in the fiber protein of adenovirus belonging to Group B, more preferably at least the fiber knob region in the fiber protein of adenovirus of any type selected from the group consisting of type 34, type 35, type 3, type 7, type 11, type 16, type 21 and type 50, and even more preferably at least the fiber knob region in the fiber protein of adenovirus type 34 or 35. Moreover, the technical idea of the present invention is not limited to these fiber proteins as long as the intended protein binds to CD46, and it also covers various proteins capable of binding to CD46 as well as proteins having a motif capable of binding to CD46.

Alternatively, in the present invention, the CD46-binding fiber protein may comprise a region consisting of the fiber knob region and the fiber shaft region in the fiber protein of adenovirus belonging to Group B, more preferably a region consisting of the fiber knob region and the fiber shaft region in the fiber protein of adenovirus of any type selected from the group consisting of type 34, type 35, type 3, type 7, type 11, type 16, type 21 and type 50, and even more preferably a region consisting of the fiber knob region and the fiber shaft region in the fiber protein of adenovirus type 34 or 35.

In the present invention, the CD46-binding fiber protein may comprise the fiber shaft region or the fiber tail region in the fiber protein of adenovirus of any type (e.g., type 2, type 5) other than the above types, as long as it comprises at least the fiber knob region in the fiber protein of adenovirus belonging to Group B.

Examples of such a fiber protein include, but are not limited to, fiber proteins which comprise a region consisting of not only the fiber knob region and the fiber shaft region in the fiber protein of adenovirus of any type selected from the group consisting of type 34, type 35, type 3, type 7, type 11, type 16, type 21 and type 50, but also the fiber tail region in the fiber protein of adenovirus type 5.

The nucleotide sequences of a gene encoding the fiber knob region in the fiber protein of adenovirus type 34, a gene encoding the fiber shaft region in the fiber protein of adenovirus type 34 and a gene encoding a region consisting of the fiber knob region and the fiber shaft region in the fiber protein of adenovirus type 34 are shown in SEQ ID NOs: 47, 48 and 49, respectively.

Likewise, the nucleotide sequence of a gene encoding a region consisting of not only the fiber knob region and the fiber shaft region in the fiber protein of adenovirus type 34, but also the fiber tail region in the fiber protein of adenovirus type 5 is shown in SEQ ID NO: 50. In the present invention, the nucleotide sequence of such a gene includes not only the nucleotide sequence shown in SEQ ID NO: 50, but also nucleotide sequences which are hybridizable under stringent conditions with DNA consisting of a nucleotide sequence complementary to DNA consisting of the nucleotide sequence shown in SEQ ID NO: 50 and which encode a protein with binding activity to CD46. Procedures for evaluation of the binding activity to CD46, procedures and stringent conditions for hybridization are the same as described above.

To prepare the recombinant adenovirus of the present invention, a polynucleotide comprising the replication cassette, the labeling cassette and/or the cell death-inducing cassette may be excised with appropriate restriction enzymes and inserted into an appropriate virus expression vector. A preferred virus expression vector is an adenovirus vector, more preferably an adenovirus type 5 vector, and particularly preferably an adenovirus type 5 vector which comprises a gene encoding a CD46-binding adenovirus fiber protein (e.g., the fiber protein of adenovirus type 34 or 35).

As shown in Example 2 described later, GFP expression in blood cells was sufficiently suppressed in both cases where a miRNA target sequence was inserted downstream of the replication cassette and where a miRNA target sequence was inserted downstream of the labeling cassette, whereas GFP expression in blood cells was unexpectedly significantly suppressed in a case where miRNA target sequences were simultaneously inserted downstream of the replication cassette and downstream of the labeling cassette, respectively. This is a new finding in the present invention.

In the present invention, the recombinant adenovirus may be obtained in the following manner, by way of example.

First, pHMCMV5 (Mizuguchi H. et al., Human Gene Therapy, 10; 2013-2017, 1999) is treated with restriction enzymes and a target sequence of miRNA is inserted to prepare a vector having the target sequence of miRNA. Next, pSh-hAIB comprising a construct of hTERT promoter-E1A-IRES-E1B (WO2006/036004) is treated with restriction enzymes and the resulting fragment comprising the hTERT promoter-E1A-IRES-E1B construct is inserted into the above vector having the target sequence of miRNA to obtain a vector comprising hTERT promoter-EIA-IRES-E1B-miRNA target sequence. On the other hand, pHMCMVGFP-1 (pHMCMV5 comprising EGFP gene) is treated with restriction enzymes to obtain a fragment comprising CMV promoter and EGFP gene, and this fragment is inserted into the above vector having the target sequence of miRNA to obtain a vector comprising a construct of CMV-EGFP-miRNA target sequence. Then, the vector comprising hTERT promoter-EIA-IRES-E1B-miRNA target sequence and the vector comprising CMV-EGFP-miRNA target sequence are each treated with restriction enzymes and ligated together to obtain a vector in which hTERT promoter-E1A-IRES-E1B-miRNA target sequence is integrated into the E1-deficient region of the adenovirus genome and CMV-EGFP-miRNA target sequence is integrated into the E3-deficient region of the adenovirus genome. Alternatively, when a vector comprising a gene encoding a CD46-binding adenovirus fiber protein is used as a vector to be inserted with the DNA fragments comprising the respective constructs, it is possible to obtain a vector in which hTERT promoter-EIA-IRES-E1B-miRNA target sequence is integrated into the E1-deficient region of the adenovirus genome and CMV-EGFP-miRNA target sequence is integrated into the E3-deficient region of the adenovirus genome and which comprises a gene encoding a CD46-binding adenovirus fiber protein. Moreover, this vector may be linearized with a known restriction enzyme and then transfected into cultured cells (e.g., 293 cells) to thereby prepare an infectious recombinant adenovirus. It should be noted that those skilled in the art would be able to easily prepare all viruses falling within the present invention by making minor modifications to the above preparation procedures.

3. Reagent for Cancer Cell Detection or Reagent for Cancer Diagnosis

As described above, the recombinant adenovirus of the present invention has the following features.

(i) This recombinant adenovirus infects almost all cells except for erythrocytes, and is also able to infect highly malignant CAR-negative cancer cells.
(ii) This recombinant adenovirus grows specifically in hTERT-expressing cancer cells and also increases the expression level of a reporter gene upon growth, whereby the production of a labeling protein, a chromophore or the like can be increased to detectable levels.
(iii) This recombinant adenovirus can prevent the occurrence of false positive results even when the virus infects normal cells having hTERT promoter activity, because miRNA expression prevents not only growth of the virus, but also expression of a reporter gene. In particular, because of comprising a target sequence of miRNA which is expressed specifically in blood cells, this recombinant adenovirus can prevent the occurrence of false positive results even when the virus infects normal blood cells having hTERT promoter activity, because expression of this miRNA prevents not only growth of the virus in blood cells but also expression of a reporter gene.

Thus, the recombinant adenovirus of the present invention can be used as a reagent for cancer cell detection or as a reagent for cancer diagnosis. In particular, because of having the above features, the recombinant virus of the present invention is extremely effective for detection of circulating tumor cells (CTCs) present in blood.

On the other hand, since 2004 when CTCs, which are cancer cells present in blood, were reported to serve as a prognostic factor for post-operative breast cancer patients in the New England Journal of Medicine (Cristofanilli M. et al., The New England Journal of Medicine, 2004, 781-791), CTCs have been measured as a biomarker in many clinical trials conducted in Europe and North America. Particularly in breast cancer, prostate cancer and skin cancer, CTCs have been proven to be an independent factor which determines the prognosis of these cancers. Moreover, in Europe, in the clinical trial in adjuvant setting of prostate cancer (SUCCESS), the number of CTCs counted is added to the inclusion criteria and only patients in whom one or more cells have been detected are included. This trial is a large-scale clinical trial including 2000 cases or more, and attention is being given to the results. Moreover, there is also a clinical trial in which an increase or decrease per se in CTCs is one of the clinical endpoints (MDV3100).

In recent years, the FDA in the United States has issued guidelines for approval and authorization of molecular-targeted anticancer agents, and hence the CTC test has become more important in cancer diagnosis. The guidelines issued by the FDA define that genetic changes in molecular targets in tumors should be tested before selection of molecular-targeted anticancer agents. When attempting to achieve the guidelines by conventional techniques, there arises a need for surgical biopsy from tumor tissues in patients to conduct genetic testing, which will impose a very strong burden on the patients. To solve this problem, efforts are now made to conduct genetic testing on CTCs collected from blood, and this strategy is referred to as “liquid biopsy” in contrast to the conventional “biopsy.” Once this strategy has been achieved, genetic testing of tumor tissues can be conducted simply by blood collection and the burden on patients can be reduced greatly. For these reasons, the CTC test is receiving great attention as a highly useful testing technique in the clinical setting.

The CellSearch System of Veridex LLC is the only CTC detection device currently approved by the FDA, and most of the CTC detection methods used in clinical trials are accomplished by this CellSearch System. The CellSearch System is based on techniques to detect cancer cells with EpCAM antibody and cytokeratin antibody.

However, CTC detection techniques are designed to detect several to several tens of cells from among a billion of blood cells, and it is therefore very difficult to improve their sensitivity and accuracy. Thus, some problems are also pointed out in CTC detection methods based on the CellSearch System. For example, it is pointed out that cancer cells which are negative in the CTC test based on the CellSearch System are detected as being positive in another test, and that there are great differences in sensitivity and accuracy, depending on the cancer type (Allard W. J. et al., Clinical Cancer Research, 2004, 6897-6904). Moreover, the CellSearch System is also pointed out to have a problem of low CTC detection rate for lung cancer in the clinical setting (ibid).

Likewise, the CellSearch System is also pointed out to have a problem of reduced CTC detection rate because the expression of cell surface antigens including EpCAM is reduced in cancer cells having undergone epithelial-mesenchymal transition (EMT) (Anieta M. et. al., J Natl Cancer Inst, 101, 2009, 61-66, Janice Lu et. al., Int J Cancer, 126(3), 2010, 669-683).

Further, to conduct the above “liquid biopsy,” additional steps are required for concentration and phenotyping or genotyping of CTCs, which require more sensitive and more accurate CTC detection techniques than simply counting the number of CTCs.

In contrast to this, because of having the above features (i) to (iii), the recombinant adenovirus of the present invention allows simple, highly sensitive and highly accurate detection of CTCs in blood without detection of leukocytes and other normal blood cells. Further, the reagent of the present invention allows detection of CTCs alive, so that the source organ of the detected CTCs can be identified upon analyzing surface antigens or the like present on the cell surface of the CTCs. Thus, the recombinant adenovirus of the present invention is useful for CTC detection and cancer diagnosis.

Moreover, the recombinant adenovirus or reagent for cancer cell detection of the present invention can be used to detect cancer cells having undergone EMT or mesenchymal-epithelial transition (MET). EMT is a phenomenon in which cancer cells lose their properties as epithelium and acquire features as mesenchymal lineage cells tending to migrate into surrounding tissues, and EMT is also involved in invasion and/or metastasis of cancer cells. On the other hand, mesenchymal-epithelial transition (MET) is a phenomenon in which mesenchymally derived cells acquire features as epithelium. As described above, it is difficult to detect cancer cells having undergone EMT by known techniques including the CellSerch System. In contrast, the present invention allows detection of cancer cells having undergone EMT or MET. The recombinant adenovirus of the present invention is therefore useful for cancer cell detection and for cancer diagnosis.

Further, the recombinant adenovirus of the present invention can also be used to detect drug-resistant cancer cells. Drugs intended in the present invention are those used for cancer chemotherapy. Examples of such drugs include, but are not limited to, adriamycin, carboplatin, cisplatin, 5-fluorouracil, mitomycin, bleomycin, doxorubicin, daunorubicin, methotrexate, paclitaxel, docetaxel and actinomycin D, etc. Moreover, the recombinant virus of the present invention can also be used to detect cancer stem cells. In the present invention, cancer stem cells refer to cells (stem cells) serving as the origin of cancer cells. Cancer stem cells also include those having drug resistance.

In the present invention, the type of cancer or tumor to be detected or diagnosed is not limited in any way, and cells of all cancer types can be used. Examples include solid cancers or blood tumors, more specifically brain tumor, cervical cancer, esophageal cancer, tongue cancer, lung cancer, breast cancer, pancreatic cancer, gastric cancer, small intestinal cancer, duodenal cancer, colorectal cancer, bladder cancer, kidney cancer, liver cancer, prostate cancer, uterine cancer, uterine cervical cancer, ovarian cancer, thyroid cancer, gallbladder cancer, pharyngeal cancer, sarcoma, melanoma, leukemia, lymphoma and multiple myeloma (MM). Most (85% or more) of the cancer cells derived from human tissues show increased telomerase activity, and the present invention allows detection of such telomerase-expressing cancer cells in general.

Moreover, in the present invention, CTCs are not limited in any way as long as they are cancer cells present in blood, and they include not only cancer cells released from solid cancers, but also blood tumor cells such as leukemia cells and lymphoma cells as mentioned above. However, in cases where CTCs are blood tumor cells, the miRNA target sequence contained in the adenovirus of the present invention is preferably a target sequence of miRNA which is expressed specifically in normal blood cells.

To prepare the reagent of the present invention, the recombinant adenovirus may be treated, e.g., by freezing for easy handling and then used directly or mixed with known pharmaceutically acceptable carriers (e.g., excipients, extenders, binders, lubricants) and/or known additives (including buffering agents, isotonizing agents, chelating agents, coloring agents, preservatives, aromatics, flavorings, sweeteners).

4. Method for Cancer Cell Detection or Method for Cancer Diagnosis

Furthermore, the recombinant adenovirus of the present invention can be used for cancer cell detection or cancer diagnosis by contacting the same with cancer cells and detecting the fluorescence or color produced by the cancer cells.

In the present invention, the term “contact(ing)” is intended to mean that cancer cells and the recombinant adenovirus of the present invention are allowed to exist in the same reaction system, for example, by adding the recombinant adenovirus of the present invention to a sample containing cancer cells, by mixing cancer cells with the recombinant adenovirus, by culturing cancer cells in the presence of the recombinant adenovirus, or by infecting the recombinant adenovirus into cancer cells. Moreover, in the present invention, “fluorescence or color” is not limited in any way as long as it is light or color produced from a protein expressed from a reporter gene, and examples include fluorescence emitted from a labeling protein (e.g., GFP), light emitted from a luminophore generated by luciferase-mediated enzymatic reaction, blue color produced from a chromophore generated by enzymatic reaction between β-galactosidase and X-gal, etc.

Cancer cells for use in the method for cancer cell detection or in the method for cancer diagnosis may be derived from a biological sample taken from a subject. Such a biological sample taken from a subject is not limited in any way as long as it is a tissue suspected to contain cancer cells, and examples include blood, tumor tissue, lymphoid tissue and so on. Alternatively, cancer cells may be circulating tumor cells (CTCs) in blood, and explanations on CTCs are the same as described above.

Cancer cell detection and cancer diagnosis using the reagent of the present invention may be accomplished as follows, by way of example.

In cases where the biological sample taken from a subject is blood, the blood sample is treated by addition of an erythrocyte lysis reagent to remove erythrocytes and the remaining cell suspension is mixed in a test tube with the reagent of the present invention at a given ratio (0.01 to 1000 MOI (multiplicity of infection), preferably 0.1 to 100 MOI, more preferably 1 to 10 MOI). The test tube is allowed to stand or rotated for culture at room temperature or 37° C. for a given period of time (e.g., 4 to 96 hours, preferably 12 to 72 hours, more preferably 18 to 36 hours) to facilitate virus infection into cancer cells and virus growth. GFP fluorescence production in the cell fraction is quantitatively analyzed by flow cytometry. Alternatively, GFP-expressing cells are morphologically analyzed by being observed under a fluorescence microscope. This system allows highly sensitive detection of CTCs present in peripheral blood. This method can be used for detection of CTCs which are present in trace amounts in peripheral blood.

In cases where flow cytometry is used for CTC detection, CTCs may be detected by determining whether each cell is GFP-positive or GFP-negative, e.g., in accordance with the following criteria.

First, groups of cells in a sample which is not infected with any virus are analyzed to obtain a background fluorescence value. A threshold is set to the maximum fluorescence value. Subsequently, groups of cells in samples which have been infected with the virus of the present invention are analyzed and groups of cells in a sample showing a fluorescence value equal to or greater than the threshold are determined to be GFP-positive. In the case of using a blood sample taken from a subject, GFP-positive cells can be detected as CTCs. Further, these GFP-positive cells (CTCs) may be concentrated for phenotyping or genotyping.

In the present invention, examples of a subject include mammals such as humans, rabbits, guinea pigs, rats, mice, hamsters, cats, dogs, goats, pigs, sheep, cows, horses, monkeys and so on.

The amount of the reagent of the present invention to be used is selected as appropriate, depending on the state and amount of a biological sample to be used for detection and the type of detection method to be used, etc. For example, in the case of a blood sample, the reagent of the present invention can be used in an amount ranging from about 0.01 to 1000 MOI, preferably 0.1 to 100 MOI, and more preferably 1 to 10 MOI per 1 to 50 ml, preferably 3 to 25 ml, and more preferably 5 to 15 ml of the blood sample. MOI refers to the ratio between the amount of virus (infectious unit) and the number of cells when a given amount of cultured cells are infected with a given amount of virus particles, and is used as an index when viruses are infected into cells.

To infect the recombinant virus into cells, the following procedures may be used for this purpose. First, cells are seeded in a culture plate containing an appropriate culture medium and cultured at 37° C. in the presence of carbon dioxide gas. The culture medium is selected from DMEM, MEM, RPMI-1640 and others commonly used for animal cell culture, and may be supplemented with serum, antibiotics, vitamins and so on, if necessary. The cultured cells are inoculated with a given amount of the virus, for example, at 0.1 to 10 MOI.

For confirmation of virus growth, the virus-infected cells are collected and treated to extract their DNA, followed by real-time PCR with primers targeting an appropriate gene possessed by the virus of the present invention, whereby virus growth can be quantitatively analyzed.

In cases where GFP gene is used as a reporter gene, labeled cells may be detected as follows: cells showing virus growth will emit a given fluorescence (e.g., a green fluorescence for GFP) upon irradiation with an excitation light, so that cancer cells can be visualized by the fluorescence. For example, when the virus-infected cells are observed under a fluorescence microscope, GFP fluorescence production can be seen in the cells. Moreover, to observe the virus-infected cells over time, GFP fluorescence production can be monitored over time with a CCD camera.

Moreover, the reagent of the present invention also allows real-time detection of cancer cells present in vivo. To label and detect cells in vivo in a real-time manner, the recombinant adenovirus of the present invention may be administered in vivo.

The reagent of the present invention may be applied directly to the affected area or may be introduced in vivo (into target cells or organs) in any known manner, e.g., by injection into vein, muscle, peritoneal cavity or subcutaneous tissue, inhalation from nasal cavity, oral cavity or lungs, oral administration, catheter-mediated intravascular administration and so on, as preferably exemplified by local injection into muscle, peritoneal cavity or elsewhere, injection into vein, etc.

When the reagent of the present invention is administered to a subject, the dose may be selected as appropriate, depending on the type of active ingredient, the route of administration, a target to be administered, the age, body weight, sex and/or symptoms of a patient, and other conditions. As a daily dose, the amount of the virus of the present invention serving as an active ingredient may usually be set to around 10⁶ to 10¹¹ PFU (plaque forming units), preferably around 10⁹ to 10¹¹ PFU, given once a day or in divided doses.

Real-time in vivo monitoring of fluorescence from cancer cells has the advantage of being used for in vivo diagnostic agents. This is useful for so-called navigation surgery and so on. Details on navigation surgery can be found in WO2006/036004.

Further, the reagent of the present invention is useful for detection of CTCs as a biomarker, and hence the reagent of the present invention can be used to determine prognosis.

For example, in cases where GFP is used as a labeling protein in the virus of the present invention, a biological sample taken from a cancer patient before being treated by any cancer therapy (e.g., chemotherapy, radiation therapy, surgical operation) and a biological sample taken at a time point after a certain period (e.g., 1 to 90 days) has passed from the treatment are each infected with the virus of the present invention. Next, GFP-positive cells contained in the sample taken before the treatment and GFP-positive cells contained in the sample taken at a certain time point after the treatment are compared for their number under the same conditions. As a result, if the number of GFP-positive cells after the treatment becomes smaller than the number of GFP-positive cells before the treatment, a determination can be made that prognosis has been improved.

The present invention will be further described in more detail by way of the following illustrative examples, which are not intended to limit the scope of the invention.

### Example 1

### Preparation of Ad34 Fiber 142-3pT

(1) Preparation of pHMCMV5-miR-142-3pT

pHMCMV5 (Mizuguchi H. et al., Human Gene Therapy, 10; 2013-2017, 1999) was treated with NotI/KpnI and the resulting fragment was ligated to a double-stranded oligo, which had been prepared by annealing the following synthetic oligo DNAs, to thereby prepare pHMCMV5-miR-142-3pT(pre).

miR-142-3pT-S1:
(SEQ ID NO: 43, each underline represents a miR-
142-3p target sequence)
5′-GGCCTCCATAAAGTAGGAAACACTACACAGCTCCATAAAGTAGGA
AACACTACATTAATTAAGCGGTAC-3′
miR-142-3pT-AS1:
(SEQ ID NO: 44, each underline represents a miR-
142-3p target sequence)
5′-CGCTTAATTAATGTAGTGTTTCCTACTTTATGGAGCTGTGTAGTGTT
TCCTACTTTATGGA-3′

Then, pHMCMV5-miR-142-3pT(pre) was treated with PacI/KpnI and the resulting fragment was ligated to a double-stranded oligo, which had been prepared by annealing the following synthetic oligo DNAs, to thereby obtain pHMCMV5-miR-142-3pT having 4 repeats of a miR-142-3p target sequence.

miR-142-3pT-S2:
(SEQ ID NO: 45, each underline represents a miR-
142-3p target sequence)
5′-TCCATAAAGTAGGAAACACTACAGGACTCCATAAAGTAGGAAACA
CTACAGTAC-3′
miR-142-3pT-AS2:
(SEQ ID NO: 46, each underline represents a miR-
142-3p target sequence)
5′-TGTAGTGTTTCCTACTTTATGGAGTCCTGTAGTGTTTCCTACTTTAT
GGAAT-3′

(2) Preparation of E1 Shuttle Plasmid pHM5-hAIB-miR-142-3pT

pSh-hAIB (WO2006/036004) was digested with I-CeuI/PmeI and the digested product was electrophoresed on an agarose gel. A band of approximately 4.5 kbp (hAIB cassette) was excised from the gel and treated with GENECLEAN II (Q-Biogene) to purify and collect a DNA fragment. The purified DNA fragment (hAIB cassette) was ligated to a fragment which had been obtained from pHMCMV5-miR-142-3pT by being digested with NheI, treated with Klenow Fragment and further digested with I-CeuI, thereby obtaining pHM5-hAIB-miR-142-3pT having hTERT promoter, E1A gene, IRES (internal ribosomal entry site) sequence, E1B gene and a miR-142-3pT target sequence.

(3) Preparation of E3 Shuttle Plasmid pHM13CMV-EGFP-miR-142-3pT

pEGFP-N1 (Clontech) was digested with ApaI and NotI, and the resulting digested product was inserted into the ApaI/NotI site of pHMCMV5 to obtain pHMCMVGFP-1. pHMCMVGFP-1 was digested with PmeI/HindIII, and the digested product was electrophoresed on an agarose gel. A band of approximately 750 bp (EGFP) was excised from the gel and treated with GENECLEAN II to purify and collect a DNA fragment. The purified DNA fragment (EGFP) was ligated to a fragment which had been obtained from pBluescriptII KS+ by being digested with HincII/HindIII, thereby preparing pBSKS-EGFP. pBSKS-EGFP was digested with ApaI/XbaI, and the digested product was electrophoresed on an agarose gel. A band of approximately 750 bp (EGFP) was excised from the gel and treated with GENECLEAN II to purify and collect a DNA fragment. The purified DNA fragment (EGFP) was ligated to a fragment which had been obtained from pHMCMV5-miR-142-3pT by being digested with ApaI/XbaI, thereby obtaining pHMCMV5-EGFP-miR-142-3pT. pHMCMV5-EGFP-miR-142-3pT was digested with BglII, and the digested product was electrophoresed on an agarose gel. A band of approximately 2 kbp (CMV-EGFP-miR-142-3pT) was excised from the gel and treated with GENECLEAN II to purify and collect a DNA fragment. The purified DNA fragment (CMV-EGFP-miR-142-3pT) was ligated to a fragment which had been obtained from pHM13 (Mizuguchi et al., Biotechniques, 30; 1112-1116, 2001) by being digested with BamHI and treated with CIP (Alkaline Phosphatase, Calf Intest), thereby obtaining pHM13CMV-EGFP-miR-142-3pT.

(4) Preparation of pAdHM49-hAIB142-3pT-CG 1 42-3pT

pAdHM49 (Mizuguchi et al, J. Controlled Release 110; 202-211, 2005) was treated with I-CeuI/PI-SceI and the resulting fragment was ligated to pHM5-hAIB-miR-142-3pT which had also been treated with 1-CeuI/PI-SceI, thereby preparing pAdHM49-hAIB142-3pT in which hTERT promoter, E1A gene, IRES sequence, E1B gene and a miR-142-3pT target sequence were integrated into the E1-deficient region of the Ad vector. pAdHM49 is a recombinant adenovirus in which a region covering genes encoding the fiber knob and fiber shaft of the adenovirus type 5 fiber is replaced with a region covering genes encoding the fiber knob and fiber shaft of the adenovirus type 34 fiber, and hence pAdHM49 comprises the nucleotide sequence (SEQ ID NO: 49) of a gene encoding a region consisting of the fiber knob region and the fiber shaft region in the fiber protein of adenovirus type 34. The nucleotide sequence of a gene encoding the pAdHM49 fiber protein (i.e., the fiber knob region and fiber shaft region of the adenovirus type 34 fiber and the fiber tail region of the adenovirus type 5 fiber) is shown in SEQ ID NO: 50. In the nucleotide sequence shown in SEQ ID NO: 50, the nucleotide sequence of a gene encoding the fiber tail region of the adenovirus type 5 fiber is located at nucleotides 1 to 132, the nucleotide sequence of a gene encoding the fiber shaft region of the adenovirus type 34 fiber is located at nucleotides 133 to 402, and the nucleotide sequence of a gene encoding the fiber knob region of the adenovirus type 34 fiber is located at nucleotides 403 to 975. Namely, in the nucleotide sequence shown in SEQ ID NO: 50, the nucleotide sequence of a region derived from the adenovirus type 5 fiber is located at nucleotides 1 to 132, while the nucleotide sequence of a region derived from the adenovirus type 34 fiber is located at nucleotides 133 to 975.

Then, pAdHM49-hAIB142-3pT was digested with Csp45I and the resulting fragment was ligated to a fragment which had been obtained from pHM13CMV-EGFP-miR-142-3pT by being digested with ClaI, thereby obtaining pAdHM49-hAIB142-3pT-CG142-3pT in which hTERT promoter, E1A gene, IRES sequence, E1B gene and a miR-142-3pT target sequence were integrated into the E1-deficient region of the adenovirus vector and CMV promoter, EGFP and a miR-142-3pT target sequence were integrated into the E3-deficient region of the adenovirus vector, and which further comprised a gene encoding the fiber protein of adenovirus type 34.

(5) Preparation of Ad34 Fiber 142-3pT(E1,E3)

pAdHM49-hAIB142-3pT-CG142-3pT was linearized by being cleaved with a restriction enzyme PacI whose recognition site was present at each end of the adenovirus genome therein, and the linearized product was transfected into 293 cells seeded in a 60 mm culture dish by using Lipofectamine 2000 (Invitrogen). After about 2 weeks, a recombinant adenovirus Ad34 fiber 142-3pT(E1,E3) was obtained (FIG. 1).

### Example 2

### Activity Measurement of Ad34 Fiber 142-3pT(E1,E3)

### (1) Cells

HeLa (derived from human uterine cancer cells) and LN319 (derived from human glioma cells) were used as CAR-positive cells, while LNZ308 (derived from human glioma cells), LN444 (derived from human glioma cells) and K562 (derived from human myelogenous leukemia cells) were used as CAR-negative cells. K562 cells are expressing miR-142-3p. DMEM (10% FCS, supplemented with antibiotics) was used for HeLa, LN319, LNZ308 and LN444 cells, while RPMI-1640 medium (10% FCS, supplemented with antibiotics) was used for K562 cells. These cells were cultured at 37° C. under saturated vapor pressure in the presence of 5% CO₂.

(2) Activity Measurement of Ad34 Fiber 142-3pT(E1,E3) by Flow Cytometry

Cells of each line were seeded in a 24-well plate at 5×10⁴ cells/500 ul/well and treated with Ad34 fiber 142-3pT(E1,E3) at an MOI of 10. As a control, TelomeScan (i.e., a conditionally replicating adenovirus comprising hTERT promoter, E1A gene, IRES sequence and E1B gene integrated in this order into the E1-deficient site of adenovirus type 5 and comprising CMV promoter and GFP integrated in this order into the E3-deficient site of adenovirus type 5) was used. After culture for 24 hours, the cells were collected and the number of GFP-positive cells was measured using a flow cytometer MACSQuant (Miltenyi Biotec).

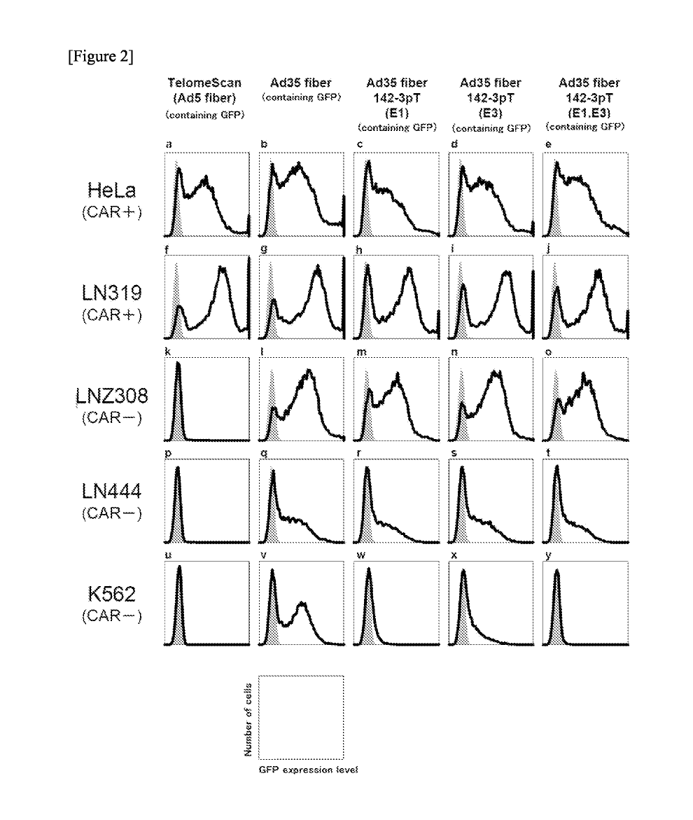

The results obtained are shown in FIG. 2. In the specification and FIG. 2, “TelomeScan (Ad5 fiber)” represents TelomeScan, while “Ad34 fiber” represents a recombinant adenovirus which comprises hTERT promoter, E1A gene, IRES sequence and E1B gene integrated in this order into the E1-deficient site of the adenovirus genome and also comprises CMV promoter and GFP integrated in this order into the E3-deficient site of the adenovirus genome and which comprises a gene encoding a fiber protein derived from adenovirus type 34. Likewise, “Ad34 fiber 142-3pT(E1)” represents a recombinant adenovirus which further comprises a target sequence of miR-142-3p integrated into the E1-deficient region (downstream of the E1B gene) in the above Ad34 fiber, while “Ad34 fiber 142-3pT(E3)” represents a recombinant adenovirus which further comprises a target sequence of miR-142-3p integrated into the E3-deficient region (downstream of the GFP gene) in the above Ad34 fiber. Likewise, “Ad34 fiber 142-3pT(E1,E3)” represents a recombinant adenovirus which further comprises a target sequence of miR-142-3p integrated into each of the E1- and E3-deficient regions (downstream of the E1B gene and downstream of the GFP gene, respectively) in the above Ad34 fiber. Moreover, in FIG. 2 and the subsequent figures, “(containing GFP)” is intended to mean that the GFP gene is inserted into each viral genome.

As a result of activity measurement, when LNZ308, LN444 and K562, which are CAR-negative cells, were infected with TelomeScan (Ad5 fiber), no GFP-positive cell was detected (FIG. 2, panels k, p and u). In contrast, when these cells were infected with Ad34 fiber, GFP-positive cells were detected (85.5% positive in LNZ308, 58.4% positive in LN444, and 63.7% positive in K562) (panels 1, q and v).

This result indicated that the recombinant adenovirus of the present invention having a gene encoding the fiber protein of adenovirus type 34 allowed significant detection of CAR-negative cells.

Further, in the case of K562 cells which are CAR-negative and are expressing miR-142-3p, GFP-positive cells were 63.7% upon infection with Ad34 fiber (panel v), whereas GFP-positive cells were 12.2% upon infection with Ad34 fiber 142-3pT(E1) and 34.8% upon infection with Ad34 fiber 142-3pT(E3), and no GFP-positive cell was detected upon infection with Ad34 fiber 142-3pT(E1,E3) (panels w, x and y). Namely, the detection rate of K562 cells was significantly reduced when using an adenovirus comprising a target sequence of miR-142-3p integrated into either the E1- or E3-deficient region of the adenovirus genome, and K562 cells were no longer detected when using an adenovirus comprising a target sequence of miR-142-3p integrated into each of the E1- and E3-deficient regions.

This result indicated that the recombinant virus of the present invention comprising a target sequence of miR-142-3p did not detect highly miR-142-3p-expressing cells, such as normal blood cells.

### Example 3

### Detection of Cancer Cells in Blood Samples Using Ad34 Fiber 142-3pT(E1,E3)

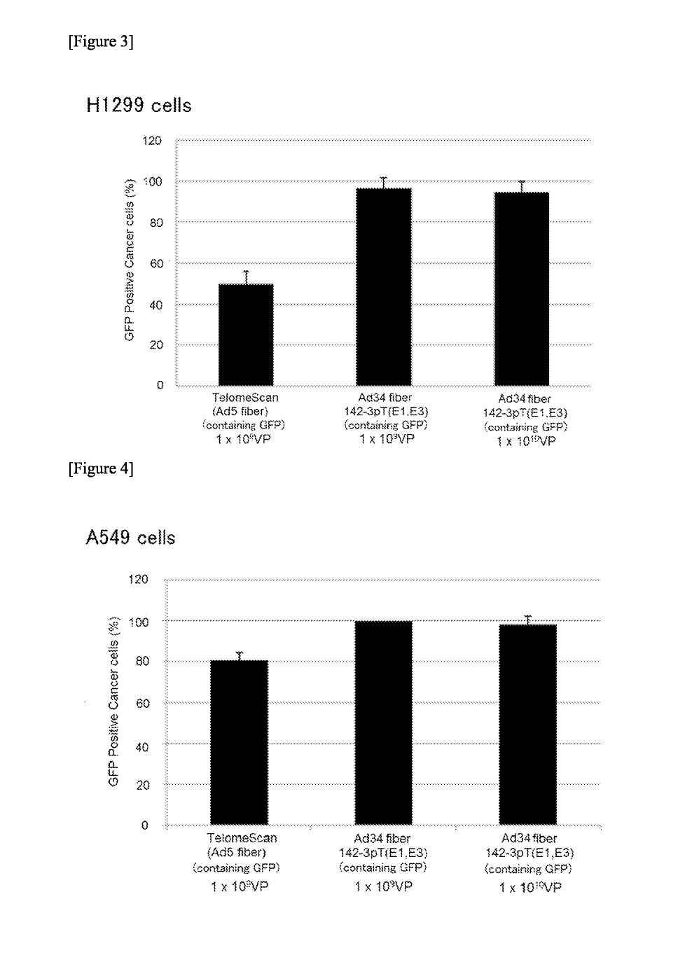

5×10⁴ H1299 cells (CAR-positive) were suspended in 5 mL blood and erythrocytes were lysed to collect PBMCs. To these PBMCs, a virus was added in an amount of 1×10⁹, 1×10¹⁰ or 1×10¹¹ VPs (virus particles) and infected at 37° C. for 24 hours while rotating with a rotator. The cells were collected and immunostained with anti-CD45 antibody, and GFP-positive cells were observed under a fluorescence microscope. CD45 is known to be a surface antigen of blood cell lineage cells except for erythrocytes and platelets. “GFP Positive Cancer cells (%)” found in the vertical axis of FIGS. 3 and 4 represents the “number of GFP-positive and CD45-negative cells (%) among GFP-positive cells.”

As a result, many false positive cells (GFP-positive and CD45-positive cells) were observed upon infection with TelomeScan (Ad5 fiber), whereas false positive cells were very few upon infection with Ad34 fiber 142-3pT(E1,E3), so that cancer cells were able to be specifically detected.

Moreover, as a result of quantitative analysis on the detection specificity of H1299 cells, many false positive cells were detected in the case of TelomeScan (Ad5 fiber) upon virus infection at 1×10⁹ VPs, whereas the detection specificity was 90% or higher and some samples showed 100% detection specificity in the case of Ad34 fiber 142-3pT(E1,E3) even when the amount of virus infection was increased (FIG. 3). Likewise, quantitative analysis was also performed on A549 cells (CAR-positive cells) in the same manner, indicating that the detection specificity was 100% upon virus infection at 1×10⁹ VPs (FIG. 4). These results indicated that the recombinant virus of the present invention allowed specific detection of cancer cells contained in the PBMC fraction.

In view of the foregoing, the detection reagent and diagnostic reagent of the present invention were demonstrated to allow detection of highly malignant CAR-negative cancer cells and, on the other hand, to ensure no false positive detection of highly miR-142-3p-expressing normal blood cells (e.g., leukocytes), etc.; and hence they were shown to be very effective for detection of circulating tumor cells (CTCs) in blood.

### Example 4

### Activity Measurement of Ad34 Fiber 142-3pT(E1,E3) in Various Human Cancer Cell Lines

### (1) Cells

The cancer cells used in this example were human non-small cell lung cancer-derived H1299 cells, human lung cancer-derived A549 cells, human breast cancer-derived MCF7 cells, human breast cancer-derived MDA-MB-231 cells, human bladder cancer-derived KK47 cells, human gastric cancer-derived MKN45 cells, human colorectal cancer-derived SW620, human liver cancer-derived Huh7 cells, human pancreatic cancer-derived Panel cells, human glioma-derived LN319 cells, human bladder cancer-derived T24 cells, human glioma-derived LNZ308 cells, and human glioma-derived LN444 cells.

(2) Activity Measurement of Ad34 Fiber 142-3pT(E1,E3) by Flow Cytometry

5×10⁴ cancer cells of each line were suspended in 500 μl medium, to which 100 μl of a conditionally replicating Ad suspension prepared at 5×10⁵ or 5×10⁶ pfu/ml was then added. The resulting mixture of the cells and the conditionally replicating Ad was seeded in a 24-well plate and cultured at 37° C. for 24 hours. The cells were collected and centrifuged at 1500 rpm for 5 minutes. After removal of the medium, the cells were suspended in 300 μl of 2% FCS-containing PBS and measured for GFP-positive rate using a flow cytometer (MACS Quant Analyzer; Miltenyi Biotec). The data obtained were analyzed by FCS multi-color data analysis software (Flowjo).

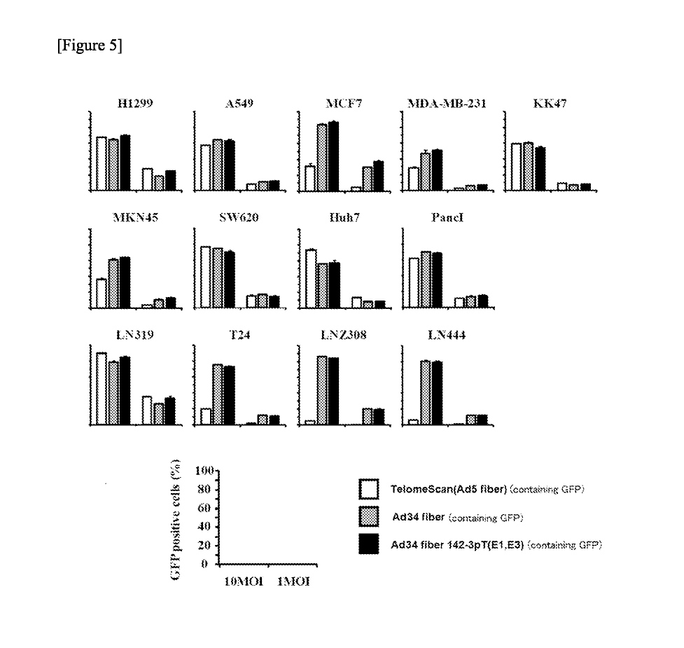

As a result, Ad34 fiber 142-3pT(E1,E3) was found to efficiently infect almost all cancer cells, and 60% or more of the cancer cells were GFP-positive. Particularly in the case of CAR-negative cells (T24, LNZ308, LN444), their GFP-positive rate was significantly improved when compared to conventionally used TelomeScan (FIG. 5).

This result indicated that the recombinant virus of the present invention allowed efficient detection of not only CAR-positive cells but also CAR-negative cells.

### Example 5

### Detection of Cancer Cells Having Undergone Epithelial-Mesenchymal Transition (EMT)

Human pancreatic cancer Panel cells were cultured for 6 days in the presence of 10 ng/mL recombinant TGF-β1 to thereby induce epithelial-mesenchymal transition (EMT). After induction of EMT, relative expression of mRNAs encoding E-cadherin, EpCAM, hTERT, N-cadherin, Slug and Snail was measured by real-time RT-PCR. In addition, CAR and CD46 expression in the Panc I cells was analyzed by flow cytometry. The virus of the present invention was infected into the cells in the same manner as shown in Example 4.

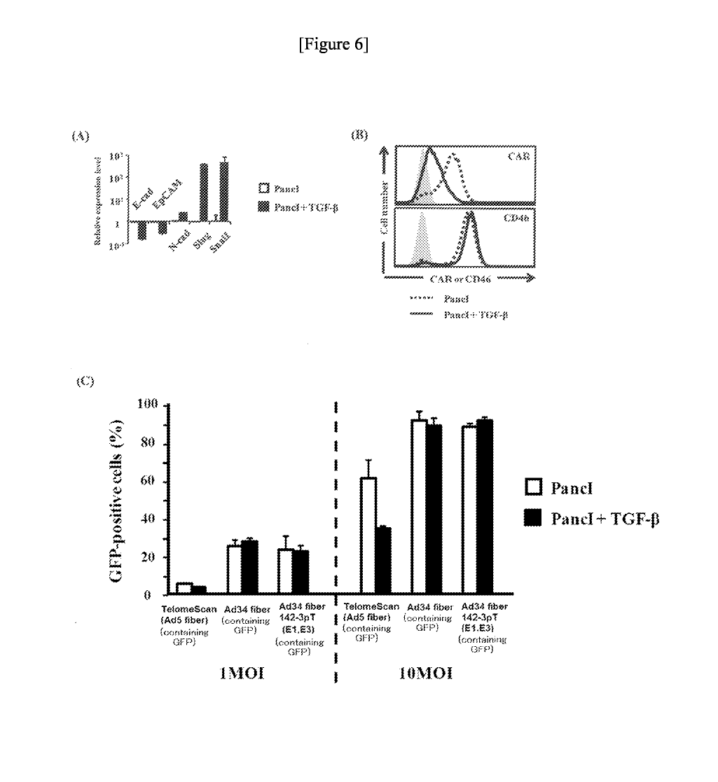

As a result, upon culture in a TGF-β-containing medium, the expression of EMT marker genes Slug, Snail and N-cadherin were increased, while the expression of epithelial markers E-cadherin and EpCAM was reduced, thus indicating that EMT has been induced (FIG. 6A). Moreover, upon EMT induction, CAR expression was reduced whereas CD46 expression was not reduced at all (FIG. 6B). Further, when conventionally used TelomeScan was used for Panel cells having undergone EMT, only about 35% of these cells were GFP-positive, whereas almost 90% or more of the cells were GFP-positive in the case of Ad34 fiber 142-3pT(E1,E3) (FIG. 6C).

These results indicated that the recombinant virus of the present invention allowed highly sensitive detection of cancer cells having undergone epithelial-mesenchymal transition (EMT).

### Example 6

### Detection of Cancer Stem Cells

MCF7 cells and MCF7-ADR cells (cancer cells resistant to the anticancer agent adriamycin) were each seeded in a 96-well plate at 1×10³ cells/well, and on the following day, adriamycin was added thereto at 0.2, 1, 5, 25 or 125 μg/mL. After 24 hours from the addition of adriamycin, an AlamarBlue® cell viability reagent was used to measure cell viability (value: mean±S.D. (n=6)).

MCF7 cells and MCF7-ADR cells were also analyzed by flow cytometry for expression of CAR, CD46, P-glycoprotein (MDR), CD24 and CD44. 5×10⁵ MCF7-ADR cells were suspended in 100 μl of 2% FCS-containing PBS, and FITC-labeled mouse anti-human CD24 antibody and PE-labeled mouse anti-human CD44 antibody were each added thereto in a volume of 1 μl, followed by reaction for 1 hour on ice under light-shielded conditions. After washing with 4 ml of 2% FCS-containing PBS, the suspension was centrifuged at 1500 rpm for 5 minutes to remove the supernatant by aspiration. The cells were suspended again in 100 μl of 2% FCS-containing PBS and subjected to a cell sorter (FACS Aria II cell sorter; BD Biosciences) to sort a CD24-negative and CD44-positive cell fraction. The data obtained were analyzed by FCS multi-color data analysis software (Flowjo). In human breast cancer cells, a fraction having the characteristics of CD24-negative and CD44-positive cells is known to be cancer stem cells (Al-Hajj M., et al., Proc Natl Acad Sci USA, 100; 3983-3988, (2003)). The virus of the present invention was infected into the cells in the same manner as shown in Example 4.

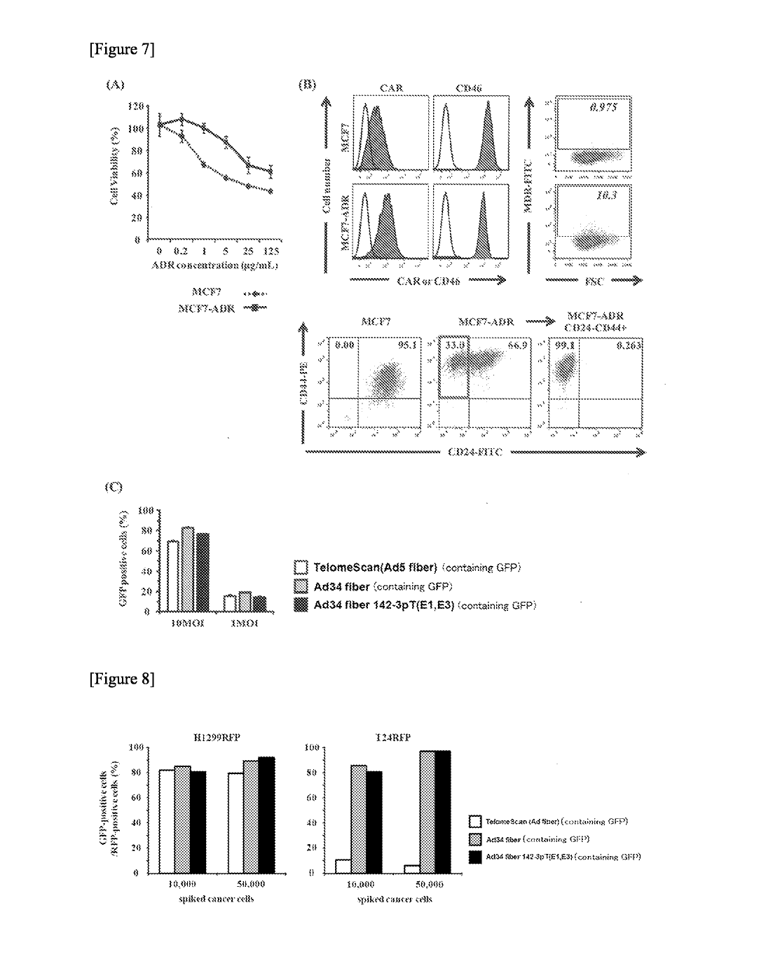

As a result, MCF7-ADR cells showed significantly high viability even in the presence of adriamycin when compared to MCF7 cells and hence were found to have drug resistance ability (FIG. 7A). MCF7-ADR cells were also found to highly express CAR and CD46 as in the case of MCF7 cells. Moreover, MCF7-ADR cells were also found to highly express MDR, which is a membrane protein responsible for drug elimination ability (FIG. 7B). Further, when Ad34 fiber 142-3pT(E1,E3) was infected into CD24-negative and CD44-positive cells among MCF-ADR cells, 80% or more of the cells were GFP-positive. In contrast, about 70% of the cells were GFP-positive in the case of conventionally used TelomeScan (FIG. 7C).

These results indicated that the recombinant virus of the present invention allowed detection of drug-resistant cancer cells. Moreover, it was also indicated that the recombinant virus of the present invention allowed detection of cancer stem cells.

### Example 7

### Detection of Cancer Cells in Blood Samples Using Ad34 Fiber 142-3pT(E1,E3)

H1299 cells or T24 cells were infected with a lentivirus vector expressing a red fluorescent protein (monomeric red fluorescent protein; RFP) at an MOI of 100 and cultured. To obtain cell clones, the cells were then seeded in a 96-well plate at 0.1 cells/well and cultured until colonies were formed. RFP-expressing cells were selected under a fluorescence microscope and subjected to extended culture, followed by flow cytometry to measure the intensity of RFP expression. Then, cells showing high intensity of RFP expression were identified as RFP-expressing cells.

Human peripheral blood mononuclear cells (hPBMCs) obtained from 1.0 mL of human peripheral blood were suspended in 800 μL of RPMI-1640 medium (10% FCS, supplemented with antibiotics). To the hPBMC suspension, cancer cells prepared at 1.0×10⁵ or 5.0×10⁵ cells/mL were added in a volume of 1004 (in FIG. 8, “spiked cancer cells” represents the number of cancer cells added to the hPBMC suspension). Further, a conditionally replicating Ad suspension prepared at 2×10⁸ pfu/mL was added in a volume of 100 μL to give a total volume of 1 mL, followed by culture at 37° C. for 24 hours while slowly rotating with a rotator.

The cell suspension cultured for 24 hours after virus infection was centrifuged at 300×g for 5 minutes to remove the supernatant. A cell fixative was added in a volume of 200 μL and reacted at 4° C. under light-shielded conditions for 15 minutes. After addition of 1 mL PBS, the suspension was centrifuged at 300×g for 5 minutes to remove the supernatant. The cells were suspended in 2% FCS-containing PBS and measured for GFP-positive rate using a flow cytometer (MACS Quant Analyzer; Miltenyi Biotec). The data obtained were analyzed by FCS multi-color data analysis software (Flowjo).

In this study, cancer cells labeled with RFP (red fluorescent protein) were mixed into hPBMCs to examine whether the cancer cells in hPBMCs were able to be detected. As a result, in the case of CAR-positive cancer cells (H1299), TelomeScan (Ad5 fiber) and Ad34 fiber 143-3pT(E1,E3) were both able to detect 80% or more of the cancer cells. On the other hand, in the case of CAR-negative cancer cells (T24), TelomeScan (Ad5 fiber) achieved very low detection efficiency (about 10% of the cells were detected as being GFP-positive), whereas Ad34 fiber 143-3pT(E1,E3) was able to detect 80% or more of the cancer cells (FIG. 8).

This result indicated that the recombinant adenovirus of the present invention allowed efficient detection of not only CAR-positive cancer cells but also CAR-negative cancer cells.

### INDUSTRIAL APPLICABILITY

Reagents comprising the recombinant adenovirus of the present invention enable simple and highly sensitive detection of CAR-negative cancer cells without detection of normal blood cells (e.g., leukocytes).

### SEQUENCE LISTING FREE TEXT

SEQ ID NO: 4: synthetic DNA
SEQ ID NOs: 5 to 26: synthetic RNA
SEQ ID NOs: 27 to 28: synthetic DNA
SEQ ID NOs: 43 to 46: synthetic DNA
SEQ ID NO: 50: synthetic DNA

1. A polynucleotide, which comprises human telomerase reverse transcriptase promoter, E1A gene, IRES sequence and E1B gene in this order and which comprises a target sequence of a first microRNA.

2. The polynucleotide according to claim 1, wherein the first microRNA is expressed in non-cancer cells.

3. The polynucleotide according to claim 1 or 2, wherein the first microRNA is at least one selected from the group consisting of miR-142, miR-15, miR-16, miR-21, miR-126, miR-181, miR-223, miR-296, miR-125, miR-143, miR-145, miR-199 and let-7.

4. A recombinant adenovirus, which comprises a replication cassette comprising the polynucleotide according to claim 1, wherein the replication cassette is integrated into the E1 region of the adenovirus genome.

5. The recombinant adenovirus according to claim 4, which further comprises a labeling cassette comprising a reporter gene and a promoter capable of regulating the expression of the gene, wherein the labeling cassette is integrated into the E3 region of the adenovirus genome.

6. The recombinant adenovirus according to claim 5, wherein the labeling cassette further comprises a target sequence of a second microRNA.

7. The recombinant adenovirus according to claim 4, wherein a cell death-inducing cassette comprising a gene encoding a cell death induction-related protein and a promoter capable of regulating the expression of the gene is further integrated into the E3 region of the adenovirus genome.

8. The recombinant adenovirus according to claim 7, wherein the cell death-inducing cassette further comprises a target sequence of a second microRNA.

9. The recombinant adenovirus according to claim 6 or 8, wherein the second microRNA is expressed in non-cancer cells.

10. The recombinant adenovirus according to claim 9, wherein the second microRNA is at least one selected from the group consisting of miR-142, miR-15, miR-16, miR-21, miR-126, miR-181, miR-223, miR-296, miR-125, miR-143, miR-145, miR-199 and let-7.

11. The recombinant adenovirus according to claim 5 or 6, wherein the reporter gene is a gene encoding a protein which emits fluorescence or a gene encoding an enzyme protein which generates a luminophore or a chromophore upon enzymatic reaction.

12. The recombinant adenovirus according to claim 5, wherein the promoter is human telomerase reverse transcriptase promoter or cytomegalovirus promoter.

13. The recombinant adenovirus according to claim 4, which further comprises a gene encoding a CD46-binding fiber protein.

14. The recombinant adenovirus according to claim 13, wherein the CD46-binding fiber protein comprises at least the fiber knob region in the fiber protein of adenovirus type 34 or 35.

15. A reagent for cancer cell detection, which comprises the recombinant adenovirus according to claim 4.

16. A reagent for cancer diagnosis, which comprises the recombinant adenovirus according to claim 4.

17. The reagent according to claim 15, wherein the cancer cells are derived from a biological sample taken from a subject.

18. The reagent according to claim 17, wherein the biological sample is blood.

19. The reagent according to claim 15 or 18, wherein the cancer cells are circulating tumor cells.

20. The reagent according to claim 15, wherein the cancer cells are drug-resistant cancer cells.

21. The reagent according to claim 15, wherein the cancer cells are cancer stem cells.

22. The reagent according to claim 15, wherein the cancer cells are cancer cells having undergone epithelial-mesenchymal transition or mesenchymal-epithelial transition.

23. A method for cancer cell detection, which comprises contacting cancer cells with the recombinant adenovirus according to claim 11 and detecting the fluorescence or color produced by the cancer cells.

24. The method according to claim 23, wherein the cancer cells are derived from a biological sample taken from a subject.

25. The method according to claim 24, wherein the biological sample is blood.

26. The method according to claim 25, wherein the cancer cells are circulating tumor cells.
